(19) United States
(12) Patent Application Publication
(10) Pub. No.: US 2005/0032866 A1
(43) Pub. Date: Feb. 10, 2005

(54) The use of 5ht4 receptor antagonists in the prophylaxis or treatment of certain cardiovascular conditions

(76) Inventors: Mireille Marguerite Jeanne Bonhomme, Les Ulis (FR); Antoine Michel Alain Bril, Suresnes (FR); Bernard Emile Joseph Gout, Rennes (FR); Bela Rajiv Patel, King of Prusia, PA (US); Gillian Louise Shepherd, Twickenham (GB); Neeta Balkrishan Amin, King of Prusia, PA (US); Bernard Enno Ilson, Philadelphia, PA (US)

Correspondence Address: SMITHKLINE BEECHAM CORPORATION;CORPORATE INTELLECTUAL PROPERTY-US, UW2220, P. O. BOX 1539, KING OF PRUSSIA, PA, 19406-0939, US

(21) Appl. No.: 10/344,075

(22) PCT Filed: Aug. 7, 2001
(86) PCT No.: PCT/GB01/03544

(30) Foreign Application Priority Data
Aug. 7, 2000 (GB) 0019410.0
Aug. 8, 2000 (GB) 0019523.0
Aug. 8, 2000 (GB) 0019524.8
Aug. 2, 2001 (GB) 0118919.0
Aug. 3, 2001 (GB) 0119022.2

(51) Int. Cl. A61K 9/00; A61K 45/00

(52) U.S. Cl. 514/400; 424/400

(57) ABSTRACT

The invention relates to the use of a 5-HT₄ receptor antagonist in the manufacture of a medicament for the prophylaxis or treatment of atrial remodelling in a mammal. Preferably, the antagonist is N-[(1-ⁿbutyl-4-piperidinyl)methyl]-3,4-dihydro-2H-[1,3]oxazino[3,2-a]indole-10-carboxamide (SB 207266) or a pharmaceutically acceptable salt thereof. The invention also relates to the use of SB 207266 or a pharmaceutically acceptable salt thereof in the manufacture of a medicament for the treatment or prophylaxis of atrial fibrillation in a mammal by administering to the mammal a daily oral or parenteral dosage regimen of about 0.2 mg to 1.0 mg of the SB 207266 or salt thereof per kg of total body weight (measured as the free base). The invention also relates to the use of SB 207266 or a pharmaceutically acceptable salt thereof in the prophylaxis or treatment of atrial arrhythmia in a mammal by administration of the SB 207266 or salt thereof on the first day at a loading dose of about 1.2 to about 2.0 times the daily maintainance dose, followed by administration of the SB 207266 or salt at the daily maintainance dose on subsequent days.

This invention relates to the use of certain compounds in the treatment or prophylaxis of certain cardiovascular conditions such as atrial remodelling, and to the use of the compounds in the treatment or prophylaxis of atrial fibrillation using specified dosages and/or dosage regimens.

### INTRODUCTION

Atrial fibrillation (AF) is the most often met arrhythmia in the clinical setting. It is a major risk for embolic stroke and is associated with an increase in mortality risk. AF (whose symptoms can include palpitations of the atrium, etc.) is a condition started by a “trigger”, such as an atrial ectopic beat (irregular heart beat) or atrial tachycardia (flutter), interacting with a “substrate” such as abnormal atrial tissue for example having a spatial heterogeneity of refractoriness or anatomical sites of conduction block. The fibrillation consists of a wavefront of excitation travelling in a continuous circular path around the atrium. Once excited, the atrial tissue takes some time to recover to a state where it can be excited again, this time being called the “refractory period” (AERP=atrial effective refractory period). Thus if the refractory period is greater than the time for the excitation wavefront to circle through 360°, then the wavefront hits non-excitable “refractory” material and the fibrillation can stop, the heart returning to sinus rhythm. Otherwise, the AF wavelets undergo “re-entry” and the atrial fibrillation continues, sometimes almost indefinitely. Patients with paroxysmal AF often progress to chronic (persistent or permanent) AF. Indeed, interdependent with the above mentioned trigger and substrate, a facilitating factor contributes to the progression and perpetuation of the disease. The facilitator called atrial remodelling is caused by a variety of structural, cellular, electrophysiological, and neurohormonal changes (e.g. activation of sympathetic and/or renin-angiotensin systems) sometimes caused by the recurrence of AF episodes. Some antiarrythmic drugs work in part by increasing the atrial refractory period and/or by increasing or decreasing the atrial “conduction velocity”. Increasing the atrial refractory period will increase the atrial wavelength and thus decrease the number of re-entry wavelets and mitigating/reducing AF. The wavelength for circus movement re-entry=the conduction velocity×the refractory period. See Tse H F and Lau C P, Clin. Exp. Pharmacol. Physiol., May 1998, 25(5), 293-302; Lau C P and Tse H F, Clin. Exp. Pharmacol. Physiol., December 1997, 24(12), 982-3; and Janse M J, Eur. Heart. J., May 1997, 18 (Suppl. C), C12-C18 for reviews.

Studies in patients with AF have shown that structural/anatomic changes can occur in the atria which can tend to sustain AF, but the relationship between the structural remodelling and the chronicity of the arrhythmia are not well understood. The changes mainly concern adaptive (dedifferentiation of cardiomyocytes) and maladaptive (degeneration of cells with replacement fibrosis) features. (By fibrosis is meant e.g. an increase in connective tissue). Atrial dilatation and/or enlargement can also occur. These structural changes are generally, though not always, observed during prolonged sustained AF. (See Thijssen V L et al., Cardiovasc Pathol 2000 January-February; 9(1):17-28; and Janse M J, Eur Heart J 1997 May; 18 Suppl C:C12-8 for reviews). On the other hand, in the setting of sustained atrial fibrillation, significant left atrial and left atrial appendage functional and anatomical remodelling (e.g. enlargement) have been found not to occur as a result of one to two months of sustained atrial fibrillation, a duration similar to that experienced by patients undergoing warfarin anticoagulation before elective cardioversion, in one study (Weigner M J et al., Heart 1999 November; 82(5):555-8).

“Atrial remodelling” is the process in which mechanical and cellular changes in the atria (structural/anatomic changes) and/or electrophysiological (electrical) changes in the atria are generated, often as a result of the development of AF, though atrial remodelling is not always the result, i.e. is not the inevitable result, of atrial fibrillation, especially in paroxysmal AF patients. (See Thijssen V L et al., Cardiovasc. Pathol., January-February 2000, 9(1), 17-28; Tse H F and Lau C P, Clin. Exp. Pharmacol. Physiol., May 1998, 25(5), 293-302 (see especially pp. 293-295 and 299-300); Lau C P and Tse H F, Clin. Exp. Pharmacol. Physiol., December 1997, 24(12), 982-3; and Janse M J, Eur. Heart. J., May 1997, 18 (Suppl. C), C12-C18, for reviews of atrial fibrillation and atrial remodelling). These remodelling changes often tend to sustain AF. The structural/anatomic changes have been described above.

“Electrophysiological (electrical) atrial remodelling” as referred to herein includes or means a) modification (especially shortening) of the atrial effective refractory period (AERP) or atrial refractoriness, b) modification of the rate adaption of the refractory period (e.g. disappearance of the normal rate adaption, so that, following the slowing of the heart rate, the refractory period does not prolong as expected), and/or c) modification of action potentials (e.g. shortening of duration, change in configuration etc.). Preferably, electrophysiological (electrical) atrial remodelling means modification (especially shortening) of the atrial effective refractory period (AERP) or atrial refractoriness. Optionally, electrical atrial remodelling can also include modification (especially slowing) of the atrial conduction velocity and/or modification (especially an increase) in dispersion, e.g. in dispersion of refractoriness. By “dispersion” is meant the difference in the magnitude of one or more electrical phenomena such as the refractory period (e.g. AERP) between spatially close areas of the tissue.

The atrial effective refractory period (AERP), and/or a decrease or increase in AERP, can be determined by conventional techniques well known to the skilled person. For example, the AERP can be determined using the conventional single extrastimulus technique, e.g. as described in A. Bril, B. Gout et al., J. Pharmacol. Exp. Ther., 1996, 276, 637-646. According to this publication (and also Example 2 of the present patent application, hereinafter), a 8-stimulus train at a basic cycle length 20% shorter than the sinus rhythm is followed by a single premature extrastimulus (4 ms, 1.5 times threshold current) introduced at progressively shorter coupling intervals from the atrial pacing until no atrial response is obtained. The AERP represents the longest coupling interval which fails to induce a propagated response in the tissue.

For similar or alternative AERP measurement methods, see the references (e.g. references 14, 16, 17, 63, 64 and 66) cited in the review paper Tse H F and Lau C P, Clin. Exp. Pharmacol. Physiol., May 1998, 25(5), 293-302, see especially p. 299. For example, reference 66 of the Tse 1998 review (E. G. Daoud et al., Circulation., 1996, 94, 1600-1606), discloses that atrial ERP can be measured by an incremental technique in 5-ms steps at basic drive cycle lengths of 350 and 500 ms for eight beats with a 1-second pause between pacing trains; the AERP is defined as the longest S₁-S₂ coupling interval that fails to result in atrial capture; the pre-AF atrial ERP can optionally be measured three times and averaged. Alternatively, reference 63 of the Tse 1998 review (the well-cited 1995 paper of M. C. E. F. Wijffels et al., Atrial fibrillation begets atrial fibrillation: A study in awake chronically instrumented goats, Circulation, 1995, 92(7), 1954-68) and the later paper of M. C. E. F. Wijffels et al., Circulation, 1997, 96, 3710-3720 disclose in their Methods sections a method of measuring AERP (e.g. at left or right atrial appendage) during a wide range of atrial pacing frequencies (S₁S₁ pacing interval, 120 to 600 ms). In this Wijffels method, a single premature stimulus (S₂) of four times the (diastolic) threshold is interpolated after every fifth basic (S₁S₂) interval. Starting from well within the refractory period (shorter than the AERP), the S₁S₂ coupling interval is incremented in steps of 1 ms. The shortest S₁S₂ interval resulting in a propagated (premature) atrial response is taken as the AERP. This Wijffels method of measuring the refractory period is fast (usually taking <30 seconds) and reproducible/reliable because the coupling interval of the test stimulus can be incremented rapidly without disturbing the steady state of the paced heart rate.

In humans and pigs, 5-HT₄ receptors are present in the atrium (see e.g. A. J Kaumann et al., Naunyn-Schmiedeberg's Arch Pharmacol (1990), 342: 619-622; A. J. Kaumann et al., Br J Pharmacol (1990) 100: 879-885). A subtype of the 5-HT₄ receptor (5-HT_{4A}) has recently been characterized specifically in human atrium (O. Blondel et al, FEBS Letters, 412, 1997, pp. 465-474) as well as in the pig atrium. This 5-HT_{4A} receptor is not present in the ventricle. For 5-HT receptor nomenclature in general, see D Hoyer, Neuropharmacology, 1997, 36(4/5), 419.

WO 91/16045 and EP 0 526 540 B1 (SmithKline Beecham) disclose that cardiac 5-HT₄ receptor antagonists can be used in the treatment of atrial arrhythmias such as atrial fibrillation, and in reducing the occurrence of stroke. See also A. J. Kaumann, Trends Pharmacol. Sci., 1994, 15(12), 451-455; A. J. Kaumann, et al., Br. J. Pharmacol., 1994, 111 (Proc. Suppl. Jan), p.26P; S. S. Hegde et al., FASEB J., 1996, 10(12) 1398-1407; R. Pino et al., Cardiovascular Research (Netherlands), December 1998, 40(3), 516-522; A. J. Kaumann et al., Naunyn-Schniedeberg's Archives Pharmacol., 1994, 349(4), 331-337; and compare with the very recent paper of J. B. Crammer et al., Basic Res. Cardiol. (Germany), 2001, 96(1), 82-90, published after the priority date of the present application.

WO 93/18036 (SmithKline Beecham) discloses a large number of condensed indole compounds as 5-HT4 antagonists including, as Example 3 on pages 17-18, N-[(1-ⁿbutyl-4-piperidinyl)methyl]-3,4-dihydro-2H-[1,3]oxazino[3,2-a]indole-10-carboxamide (SB 207266) and its preferred hydrochloride salt (SB 207266-A). These compounds are disclosed for use in the treatment or prophylaxis of gastrointestinal, cardiovascular and CNS disorders, in particular irritable bowel syndrome. WO 93/18036 also states in the general description on pp. 6-7 in general terms that: “Specific cardiac 5-HT₄ receptor antagonists which prevent atrial fibrillation and other atrial arrhythmias associated with 5-HT would also be expected to reduce the occurrence of stroke”. See also U.S. Pat. No. 5,852,014, EP 0 884 319 A2, L. M. Gaster et al, J. Med. Chem., 1995, 38, 4760-4763 and Drugs of the Future, 1997, 22(12), 1325-1332 for the compound SB 207266, which is highly selective for the 5HT₄ receptor over other 5HT receptors. (The potency and selectivity of SB207266 is also shown by the 5HT4 receptor antagonist and selectivity test results presented later in the present patent application). For improved syntheses of SB 207266, see WO 98/07728, WO 98/11067; WO 00/03983; and WO 00/03984.

The structure of SB 207266 is as follows:

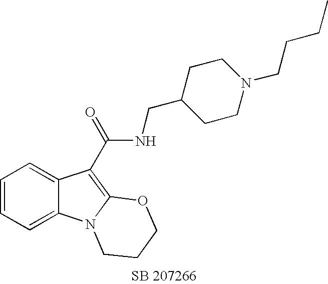

Other 5HT₄ antagonists are disclosed in WO 94/27965 (Syntex) and one of these compounds RS 100302 (Roche), whose name is N-(2-(4-(3-(8-amino-7-chloro-2,3-dihydro-1,4-benzodioxin-5-yl)-3-oxopropyl)piperidin-1-yl)ethyl)-methanesulfonamide, has been suggested to be effective in treating pig models of atrial flutter and atrial fibrillation (M. M. Rahme et al., Circulation, 1999, vol. 100(19), pp. 2010-2017). In the Rahme paper, it is noted that in the protocol on page 2011, AF or AFT was triggered in pigs by a short (60 sec) run of rapid atrial pacing without any other preparation; when AF was not induced a crush injury was made to the right atrial free wall. It is further noted that the 60 sec pacing protocol in Rahme would not have been sufficient to induce atrial remodelling, which needs several hours or days to occur depending on the mammalian species. Rapid atrial pacing, for initial sensitization, needs to be performed for about 3 to 4 hours to generate remodelling of the tissue, as described by A. Goette et al., 1996, Circulation, 94, 2968-2974, and/or as shown in the experiments shown in Examples 1 and 2 hereinafter. Similarly, the atrial crush injury disclosed in Rahme may generate physically reentrant circuits but will not generate remodelling. Rahme therefore does not disclose the use of a 5-HT₄ antagonist for the treatment of atrial remodelling.

Other 5HT₄ antagonists are disclosed in: R D Clark et al Bioorg Med Chem Lett 1994, 4(20), 2481-4; Clark, ibid, 1995, 5(18), 2119-2122 (e.g. RS100235).

WO 93/05038 (SmithKline Beecham) discloses a series of 5HT₄ antagonists, including in Example 1 the highly active and selective 5HT4 antagonist SB 204070 which is (1-butyl-4-piperidyl)methyl 8-amino-7-chloro-1,4-benzodioxan-5-carboxylate. For the hydrochloride salt of this compound (SB 204070-A) see L. M. Gaster et al, J. Med. Chem, 1993, 36, 4121-4123. Other 5HT₄ antagonists disclosed in WO 93/05038 include: SB 207710 [(1-butyl-4-piperidyl)methyl 8-amino-7-iodo-1,4-benzodioxan-5-carboxylate] and its hydrochloride as shown in Example 52; and SB 205800 [N-(1-butyl-4-piperidyl)methyl-8-amino-7-chloro-1,4-benzodioxan-5-carboxamide] as shown in Example 14. The structures of SB 204070, SB 207710, and SB 205800 are as follows:

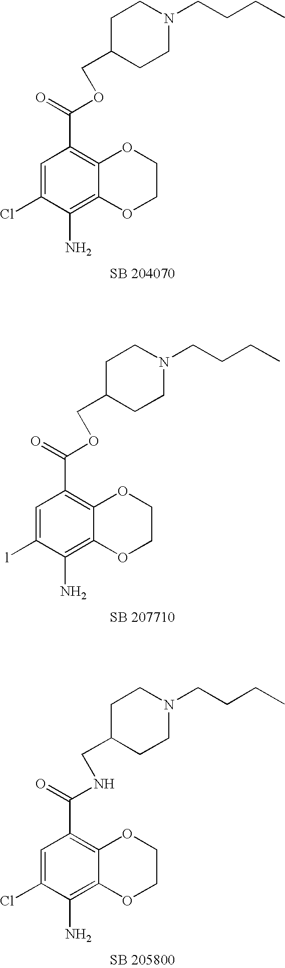

Other SmithKline Beecham publications disclosing 5HT₄ antagonists include WO 93/16072; WO 94/10174; WO 94/27987; WO 95/04737.

GR 113808—whose name is 1-methyl-1H-indole-3-carboxylic acid (1-(2-((methylsulfonyl)amino)ethyl)-4-piperidinyl)methyl ester, or alternatively [1-[2-[(Methylsulphonyl)amino]ethyl]-4-piperidinyl]methyl 1-methyl-1H-indole-3-carboxylate—is another potent and selective 5HT₄ antagonist from Glaxo Wellcome. GR 125487—whose name is [1-[2-[(Methylsulphonyl)amino]ethyl]-4-piperidinyl]methyl 5-fluoro-2-methoxy-1H-indole-3-carboxylate—is another potent and selective 5HT₄ antagonist: its pKi at 5-HT_{4A} and 5-HT_{3A} receptors=10.0 and <6.5 respectively. For GR 113808 and GR 125487, see Grossman et al, Br. J. Pharmacol., 1994, 111, 332; EP 501322 A1 and EP 501322 B1. See Example 1 of EP 501322 B1 for GR 113808 and Examples 12, 21 and 22 of EP 501322 B1 for GR 125487 and the hydrochloride, methanesulfonate and maleate salts thereof. The chemical structures of GR 113808 and GR 125487 are as follows:

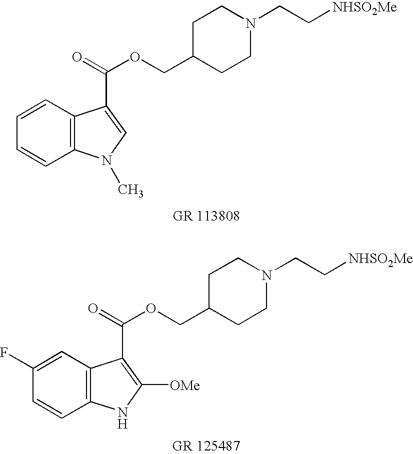

GR 138897—whose name is [1-[2-[(Methylsulphonyl)amino]ethyl]-4-piperidinyl]methyl[2-(3-methyl-1,2,4-oxadiazol-5-yl)phenyl]carbamate—is another potent and selective 5HT₄ antagonist from Glaxo Wellcome: its pKi at 5-HT_{4A} and 5-HT_{3A} receptors=10.3 and <5.0 respectively. For the synthesis of GR 138897, see Examples 1 and 3 and claims 8-10 of WO 93/20071, as well as U.S. Pat. No. 5,618,827 and EP 0 640 081 B1, and see Examples 2 and 4 and claims 9-10 of WO 93/20071 for the (Z)-2-butenedioate and methanesulfonate salts. The chemical structure of GR 138897 is as follows: 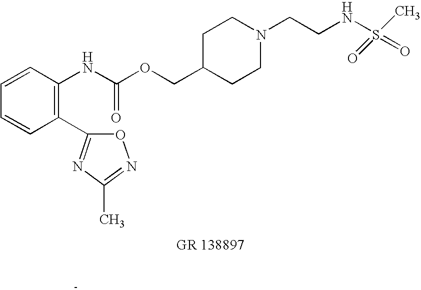
LY-353433, whose name is:
1-(1-methylethyl)-N-(2-(4-((tricyclo[3.3.1.1^{3,7}]dec-1-ylcarbonyl)amino)-1-piperidinyl)ethyl)-1H-indazole-3-carboxamide, that is:
1-(1-methylethyl)-N-(2-(4-((tricyclo[3.3.1.1 sup(3,7)]dec-1-ylcarbonyl)amino)-1-piperidinyl)ethyl)-1H-indazole-3-carboxamide, or
1-(1-methylethyl)-N-(2-(4-((tricyclo[3.3.1.1]dec-1-ylcarbonyl)amino)-1-piperidinyl)ethyl)-1H-indazole-3-carboxamide, or
N-[2-(4(1-adamantylcarbonylamino)-1-piperidinyl)ethyl]-1-(2-propyl)-1H-indazole-3-carboxamide, is a potent selective 5-HT4 antagonist being developed by Eli Lilly. See Cohen M L et al., Drug Development Research, 43: 193-199, April 1998 (including disclosure of LY 353433's active hydroxylated metabolites LY-343031 and LY-343032); Cohen M L, et al. J. Pharmacology and Experimental Therapeutics, 277: 97-104, April 1996, and see also EP 732333 A1 (e.g. see Example 27 on page 13 and claim 5 of EP 732333 A1). The structure of LY-353433 is as follows:

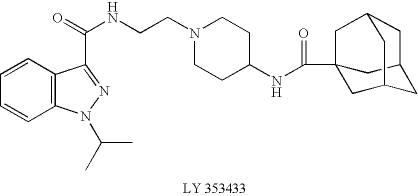

### THE INVENTION

It is desirable to discover new compounds, or classes of compounds, which can be used in the therapy (e.g. treatment or prophylaxis) of atrial remodelling.

Rapid atrial rates and/or atrial pacing, especially chronic rapid atrial rates or chronic atrial pacing (e.g. atrial pacing in an animal experimental setting), is a situation in which atrial remodelling (especially electrical remodelling) occurs in which the Atrial Effective Refractory Period (AERP) is reduced. Experimentally, such electrical remodelling is shown to play a significant role in facilitating occurrence of AF. We have now discovered that 5-HT₄ receptor antagonists (inhibitors), especially SB 207266, are capable of at least partly reversing this reduction in Atrial Effective Refractory Period (AERP), i.e. are capable of increasing the AERP. Therefore, it is expected that 5-HT₄ receptor antagonists like SB 207266 will mitigate atrial remodelling and/or protect the atria from remodelling, in particular electrical remodelling.

Thus, according to a first aspect of the invention, there is provided the use of a 5-HT₄ receptor antagonist in the manufacture of a medicament for the prophylaxis or treatment of atrial remodelling, for example in a mammal such as a human.

The invention also provides a method of treatment or prophylaxis of atrial remodelling, in a mammal (e.g. human) in need thereof, which comprises administering to said mammal an effective amount of a 5-HT₄ receptor antagonist.

The invention also provides a 5-HT₄ receptor antagonist for use in the prophylaxis or treatment of atrial remodelling, for example in a mammal such as a human.

Preferably, the invention involves the prophylaxis or treatment of electrophysiological (electrical) atrial remodelling, as defined above. More preferably, the invention involves the prophylaxis or treatment of electrical (electrophysiological) atrial remodelling, e.g. in the mammal, by increasing the atrial effective refractory period (AERP) and/or by at least partly protecting from or reversing a reduction in the AERP.

The invention, in all its aspects, can involve the prophylaxis or treatment of atrial remodelling potentiated by atrial fibrillation, for example atrial remodelling potentiated by recurrent atrial fibrillation. In all aspects of the invention, the mammal (e.g. human) treated can be a sufferer of or susceptible to atrial fibrillation, especially a sufferer [e.g. long-term (e.g. >1-year or >5-year or ≧48 hrs and ≦1 year, or ≧48 hrs and <6 month) sufferer] of persistent or permanent atrial fibrillation. Long-term (e.g. >1-year or >5-year) sufferers of persistent or permanent atrial fibrillation are more likely to have atrial remodelling problems, as discussed above. Alternatively, the mammal (e.g. human) can be a sufferer of or susceptible to paroxysmal atrial fibrillation.

Preferably or alternatively, in all aspects of the invention, the medicament/method of treatment or prophylaxis/5-HT₄ receptor antagonist (e.g. SB 207266 or a pharmaceutically acceptable salt thereof) is for, of, or for use in the inhibition (e.g. prevention) of symptomatic recurrences of atrial fibrillation in a mammal (e.g. in a human/in patients) with paroxysmal or persistent AF (preferably persistent AF). [The invention therefore also provides the use of a 5-HT₄ receptor antagonist in the manufacture of a medicament for the inhibition of symptomatic recurrences of atrial fibrillation in a mammal with paroxysmal or persistent atrial fibrillation; and/or a method of inhibiting symptomatic recurrences of atrial fibrillation in a mammal with paroxysmal or persistent atrial fibrillation, which comprises administering to said mammal an effective amount of a 5-HT₄ receptor antagonist.]

Paroxysmal, persistent and permanent AF are terms defining the severity of the AF and are understood by the skilled person.

“Paroxysmal AF” includes or means episode(s) of AF with a mean duration of individual episodes of <48 hrs. The paroxysmal AF episodes can stop spontaneously or can be converted to normal sinus rhythm (NSR) by 5HT4 antagonists and/or other antiarrhythmic drugs. The major part of paroxysmal AF is lone AF where there is no underlying cardiovascular disease and no atrial remodelling. Paroxysmal AF may turn into persistent AF if it is not terminated rapidly.

“Persistent AF”, e.g. symptomatic persistent AF, is usually longer in duration than paroxysmal AF, and includes or means episode(s), e.g. symptomatic episodes, of AF with a mean duration of individual episodes of ≧48 hrs and ≦1 year, or more preferably of mean duration ≧48 hrs and <6 months. Persistent AF does not usually spontaneously stop and usually needs electrical or pharmacological cardioversion to return to NSR. Atrial electrical remodelling often appears, left atrial enlargment can occur, as well as left ventricular dysfunction.

“Permanent AF” includes or means episode(s) of AF with a mean duration of individual episodes longer than that of persistent AF, e.g. >1-year or >5-year duration, or of permanent duration. It does not usually respond to electrical cardioversion and is associated with a profound electrical remodelling and is usually accompanied with underlying CV disease (ischemic heart disease, cardiomyopathy, and/or hypertension, etc).

Optionally, in any aspects of the invention, the invention can involve the prophylaxis or treatment of atrial remodelling potentiated by a rapid atrial rate (atrial pacing) (e.g. experimental chronic atrial pacing). The mammal (e.g. human) treated can be a sufferer of or susceptible to a rapid atrial rate (atrial pacing), e.g. an abnormally rapid atrial rate.

The 5-HT₄ receptor antagonists used in any of the aspects of the invention can include any of those referred to in the introduction. Thus, for example, the 5-HT₄ receptor antagonists used in the invention can include any compound covered by any of the claims (e.g. claim 1 et al.) of any of the patent publications referred to in the introduction as disclosing 5-HT₄ receptor antagonists (e.g. WO 93/18036, WO 93/05038, WO 93/16072; WO 94/10174; WO 94/27987; WO 95/04737; WO 93/20071, EP 501322 B1, WO 94/27965, and/or EP 732333 A1), and/or for example can include any 5-HT₄ receptor antagonist specifically exemplified in any publication (e.g. patent or journal publication) referred to in the introduction as disclosing 5-HT₄ receptor antagonist(s). As mentioned below, all publications cited in this specification, including but not limited to these 5-HT₄ receptor antagonist publications, are herein incorporated by reference as if each individual publication were specifically and individually indicated to be incorporated by reference herein as though fully set forth.

Other 5-HT₄ receptor antagonists not mentioned herein can be found using the 5-HT₄ antagonist test(s) detailed hereinafter.

Pharmaceutically acceptable salts (e.g. HCl salts), solvates, hydrates, complexes and/or prodrugs of 5-HT₄ receptor antagonists and similar derivatives are included within the scope of the definition of “5-HT₄ receptor antagonist(s)”. Suitable pharmaceutically acceptable salts will be apparent to those skilled in the art and include for example acid addition salts formed with inorganic acids eg. hydrochloric, hydrobromic, sulfuric, nitric or phosphoric acid; and organic acids eg. succinic, maleic, acetic, fumaric, citric, tartaric, benzoic, p-toluenesulfonic, methanesulfonic or naphthalenesulfonic acid.

The 5-HT₄ receptor antagonist is preferably an antagonist of the 5-HT_{4A} and/or 5-HT_{4B} receptors. The 5-HT_{4A} receptor can be as characterised in O. Blondel et al, FEBS Letters, 412, 1997, pp. 465-474. The 5-HT_{4B} receptor can be as characterised in O. Blondel et al, J. Neurochem; 1998, 70(6), pp. 2253-2261 and/or in WO 99/28456 (INSERM). Other splice variants of the 5-HT₄ receptor include 5-HT_{4C} and 5-HT_{4D}, as disclosed for example in WO 99/28456.

Preferably, the 5-HT₄ receptor antagonist is a cardiac 5-HT₄ receptor antagonist, meaning an antagonist of those 5-HT₄ receptors present in the human atrium, preferably meaning an antagonist of those 5-HT₄ receptors which in the human heart are substantially only present in the human atrium. (See e.g. Kaumann et al., Naunyn-Schmiedeberg's Arch Pharmacol (1990), 342: 619-622; A. J. Kaumann et al., Br J Pharmacol (1990) 100: 879-885, O. Blondel et al, FEBS Letters, 412, 1997, pp. 465-474; O. Blondel et al, J. Neurochem., 1998, 70(6), pp. 2253-2261; and WO 99/28456). The 5-HT_{4A} and the 5-HT_{4B} receptors are such receptors.

In fact, it has now been found by the inventors that the 5-HT_{4B} receptor is the principal 5-HT₄ receptor isoform expressed in human atria Smaller amounts of the 5-HT_{4A} and 5HT_{4C} receptor isoforms are found in human atria but 5-HT_{4D} is not detectable. Also, preliminary data suggest that there is a substantial increase in 5-HT_{4B} receptor expression in the atria of human patients with chronic (persistent) AF compared to the atria of human patients with acute (paroxysmal) AF. Also, SB 207266 has been found to be a 5-HT_{4B} antagonist. [Therefore, the invention also provides (A) the use of a 5-HT_{4B} receptor antagonist in the manufacture of a medicament for the prophylaxis or treatment of atrial remodelling and/or atrial arrhythmia (e.g. atrial fibrillation) in a human who is a sufferer of or susceptible to persistent atrial fibrillation; (B) a method of treatment or prophylaxis of atrial remodelling and/or atrial arrhythmia (e.g. atrial fibrillation), in a human in need thereof who is a sufferer of or susceptible to persistent atrial fibrillation, which comprises administering to said human an effective amount of a 5-HT_{4B} receptor antagonist; and/or (C) a 5-HT_{4B} receptor antagonist for use in the prophylaxis or treatment of atrial remodelling and/or atrial arrhythmia (e.g. atrial fibrillation) in a human who is a sufferer of or susceptible to persistent atrial fibrillation. Preferably, the antagonist is an antagonist of the human (e.g. atrial) 5-HT_{4B} receptor.]

Preferably, in any of the aspects of the invention, the 5-HT₄ (e.g. 5-HT_{4A} and/or 5-HT_{4B}) receptor antagonist is a selective 5-HT₄ (e.g. 5-HT_{4A} and/or 5-HT_{4B}) receptor antagonist. Such an antagonist may for example bind to and/or inhibit the 5-HT₄ (e.g. 5-HT_{4A} and/or 5-HT_{4B}) receptor at least 10 times, preferably at least 25 times, more preferably at least 100 times, more strongly than any other 5-HT receptor. The selectivity can be measured by known tests. See e.g. See D Hoyer, Neuropharmacology, 1997, 36(4/5), 419 and refs cited therein for 5-HT receptor nomenclature.

Preferably, the 5-HT₄ (e.g. 5-HT_{4A} and/or 5-HT_{4B}) receptor antagonist comprises a compound disclosed in the description (including the the Examples) and/or the claims of WO 93/18036. For example, in accordance with claim 1 of WO 93/18036, the 5-HT₄ receptor antagonist can comprise a compound of formula (I), or a pharmaceutically acceptable salt thereof 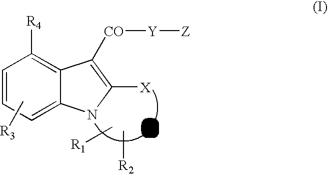
wherein
X is O, S, SO, SO₂, CH₂, CH or NR wherein R is hydrogen or C₁₋₆ alkyl;
A is a saturated or unsaturated polymethylene chain of 2-4 carbon atoms;
R₁ and R₂ are hydrogen or C₁₋₆ alkyl;
R₃ is hydrogen, halo, C₁₋₆ alkyl, amino, nitro or C₁₋₆ alkoxy;
R₄ is hydrogen, halo, C₁₋₆ alkyl or C₁₋₆ alkoxy;
Y is O or NH;
Z is of sub-formula (a), (b) or (c): 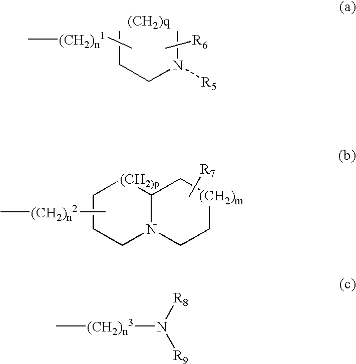
   wherein
n¹ is 1, 2, 3 or 4; n² is 0, 1, 2, 3 or 4; n³ is 2, 3, 4 or 5;
q is 0, 1, 2 or 3; p is 0, 1 or 2; m is 0, 1 or 2;
R₅ is hydrogen, C₁₋₁₂ alkyl, aralkyl or R₅ is (CH₂)_{z}—R₁₀ wherein z is 2 or 3 and R₁₀ is selected from cyano, hydroxyl, C₁₋₆ alkoxy, phenoxy, C(O)C₁₋₆ alkyl, COC₆H₅, —CONR₁₁R₁₂, NR₁₁COR₁₂, SO₂NR₁₁R₁₂ or NR₁₁SO₂R₁₂ wherein R₁₁ and R₁₂ are hydrogen or C₁₋₆ alkyl; and
R₆, R₇ and R₈ are independently hydrogen or C₁₋₆ alkyl; and
R₉ is hydrogen or C₁₋₁₀ alkyl;
   or a compound of formula (I) wherein the CO—Y linkage is replaced by a heterocyclic bioisostere.

Where the CO—Y linkage is replaced by a heterocyclic bioisostere, the bioisostere can be as disclosed on page 3 lines 11-25 of WO 93/18036. However, preferably this bioisostere is not present; i.e. preferably Y is O or NH.

Preferably, X is O. Preferably, A is —(CH₂)₃—. Preferably, R₁ and R₂ are independently hydrogen or methyl. It is preferred that R₃ is hydrogen and R₄ is hydrogen or halo. (Compare: claims 2-5 of WO 93/18036).

Aryl (for example when R₅ is aralkyl) includes phenyl and naphthyl optionally substituted by one or more substituents selected from halo, C₁₋₆ alkyl and C₁₋₆ alkoxy. When R₅ is aralkyl, this can include optionally substituted benzyl, e.g. benzyl in which the phenyl ring is substituted by one or more substituents selected from halo, C₁₋₆ alkyl and C₁₋₆ alkoxy. (Compare: claim 9 and page 3 lines 6-7 of WO 93/18036).

Preferably, Z is of sub-formula (a). In sub-formula (a), (CH₂)ₙ1 is attached at a carbon atom of the azacycle. Preferably n¹ is 1. Preferably, q=3, so that sub-formula (a) comprises a six-membered azacycle, i.e. Z is 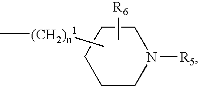
in which case preferably (CH₂)ₙ1 is attached at the 4-position of the azacycle. Still more preferably, Z is 4-piperidylmethyl N-substituted by R₅ (i.e. Z is 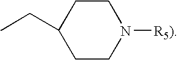
Where Z is 4-piperidylmethyl N-substituted by R₅, it is preferred that the N-substituent R₅ is C₂ or C₃ or greater alkyl (i.e. C₂₋₁₂ alkyl or C₃₋₁₂ alkyl), or optionally substituted benzyl; or the N-substituent R₅ is replaced by (CH₂)ₙR⁴ as defined in formula (I) of EP-A-501322 and in relation to the specific examples of EP-A-501322. Most preferably, Z is (1-(n-butyl)-4-piperidyl)methyl, i.e. 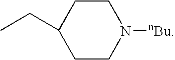
(Compare: claims 1 and 7-9 and page 4 lines 6 to page 5 line 10 of WO 93/18036).

Preferably, in the present invention, the 5-HT₄ receptor antagonist comprises a compound selected from:
(a) one of Examples 1 to 46 as described in WO 93/18036,
(b) one of Examples 1 to 54 as described in WO 93/05038,
(c) one of the compounds described in claim 6 or Examples 1 to 38 of WO 93/20071, or
(d) one of the compounds described in claim 9 or Examples 1 to 23 of EP 501322 B1,
   in free base form or as a pharmaceutically acceptable salt thereof.

Alternatively, in the present invention, the 5-HT₄ receptor antagonist can comprise a compound selected from:
(a) one of the compounds of Examples 1 to 15 as described in WO 94/27965 or RS100235 or RS100302, or
(b) one of Examples 1 to 38 as described in EP 732333 A1,
   in free base form or as a pharmaceutically acceptable salt thereof.

In the present invention, it is particularly preferred that the 5-HT₄ receptor antagonist comprises:
(i) N-[(1-ⁿbutyl-4-piperidinyl)methyl]-3,4-dihydro-2H-[1,3]oxazino[3,2-a]indole-10-carboxamide (SB 207266);
(ii) N-(2-(4-(3-(8-Amino-7-chloro-2,3-dihydro-1,4-benzodioxin-5-yl)-3-oxopropyl)piperidin-1-yl)ethyl)-methanesulfonamide (RS 100302);
(iii) 1-methyl-1H-indole-3-carboxylic acid (1-(2-((methylsulfonyl) amino)ethyl)-4-piperidinyl)methyl ester (GR 113808); or
(iv) 1-(1-methylethyl)-N-(2-(4-((tricyclo[3.3.1.1^{3,7}]dec-1-ylcarbonyl)amino)-1-piperidinyl)ethyl)-1H-indazole-3-carboxamide, that is
1-(1-Methylethyl)-N-(2-(4-((tricyclo[3.3.1.1 sup(3,7)]dec-1-ylcarbonyl)amino)-1-piperidinyl)ethyl)-1H-indazole-3-carboxamide or
1-(1-methylethyl)-N-(2-(4-((tricyclo[3.3.1.1]dec-1-ylcarbonyl)amino)-1-piperidinyl)ethyl)-1H-indazole-3-carboxamide, or
N-[2-(4-(1-adamantylcarbonylamino)-1-piperidinyl)ethyl]-1-(2-propyl)-1H-indazole-3-carboxamide (LY-353433);
   or a pharmaceutically acceptable salt thereof.

Alternatively, it is preferred that the 5-HT₄ receptor antagonist comprises:
(v) (1-butyl-4-piperidyl)methyl 8-amino-7-chloro-1,4-benzodioxan-5-carboxylate (SB 204070) or a pharmaceutically acceptable salt thereof, for example the hydrochloride salt thereof,
(vi) (1-butyl-4-piperidyl)methyl 8-amino-7-iodo-1,4benzodioxan-5-carboxylate (SB 207710) or a pharmaceutically acceptable salt thereof, for example the hydrochloride salt thereof,
(vii) N-(1-butyl-4-piperidyl)methyl-8-amino-7-chloro-1,4-benzodioxan-5-carboxamide (SB 205800) or a pharmaceutically acceptable salt thereof,
(viii) [1-[2-[(Methylsulphonyl)amino]ethyl]-4-piperidinyl]methyl[2-(3-methyl-1,2,4-oxadiazol-5-yl)phenyl]carbamate (GR 138897) or a pharmaceutically acceptable salt thereof, for example the (Z)-2-butenedioate or methanesulfonate salt thereof; or
(ix) [1-[2-[(Methylsulphonyl)amino]ethyl]-4-piperidinyl]methyl 5-fluoro-2-methoxy-1H-indole-3-carboxylate (GR 125487) or a pharmaceutically acceptable salt thereof, for example the hydrochloride, methanesulfonate or maleate salt thereof.

It is still further preferred that the 5-HT₄ receptor antagonist comprises: (i) SB 207266, (v) SB 204070, (vi) SB 207710, (vii) SB 205800, (viii) GR 138897 or (iv) LY-353433; or a pharmaceutically acceptable salt thereof. Yet further preferred is an antagonist comprising: (i), (v), (vi), (vii) or (viii) as defined above; or a pharmaceutically acceptable salt thereof.

The most preferred 5-HT₄ receptor antagonist is N-[(1-ⁿbutyl-4-piperidinyl)methyl]-3,4-dihydro-2H-[1,3]oxazino[3,2-a]indole-10-carboxamide (SB 207266) or a pharmaceutically acceptable salt thereof, in particular the hydrochloride salt thereof (SB 207266-A). SB 207266 has been found to antagonise 5HT_{4B} receptors.

A second aspect of the invention provides the use of a 5-HT₄ receptor antagonist in the manufacture of a medicament for the prophylaxis or treatment of a disease or condition, other than atrial fibrillation, associated with a reduction in atrial effective refractory period (AERP) and/or an undesirable modification of atrial refractoriness.

The second aspect of the invention also provides a method of treatment or prophylaxis of a disease or condition, other than atrial fibrillation, associated with a reduction in atrial effective refractory period (AERP) and/or an undesirable modification of atrial refractoriness, in a mammal (e.g. human) in need thereof, which comprises administering to said mammal an effective amount of a 5-HT₄ receptor antagonist.

The second aspect of the invention also provides a 5-HT₄ receptor antagonist for use in disease or condition, other than atrial fibrillation, associated with a reduction in atrial effective refractory period (AERP) and/or an undesirable modification of atrial refractoriness.

A third aspect of the invention provides the use of a 5-HT₄ receptor antagonist in the manufacture of a medicament for increasing the atrial effective refractory period (AERP) and/or beneficially modifying atrial refractoriness in a mammal (e.g. human) suffering from or susceptible to a disease or condition, other than atrial fibrillation, in which such an increase or modification is desirable.

The third aspect of the invention also provides a method of increasing the atrial effective refractory period (AERP) and/or beneficially modifying atrial refractoriness in a mammal suffering from or susceptible to a disease or condition, other than atrial fibrillation, in which such an increase or modification is desirable, in a mammal (e.g. human) in need thereof, which comprises administering to said mammal an effective amount of a 5-HT₄ receptor antagonist.

The third aspect of the invention also provides a 5-HT₄ receptor antagonist for increasing the atrial effective refractory period (AERP) and/or beneficially modifying atrial refractoriness in a mammal (e.g. human) suffering from or susceptible to a disease or condition, other than atrial fibrillation, in which such an increase or modification is desirable.

A fourth aspect provides the use of a 5-HT₄ receptor antagonist in the manufacture of a medicament for the prophylaxis or treatment of atrial pacing (e.g. chronic atrial pacing) or a disease or condition, other than atrial fibrillation, associated with episodes of atrial pacing (e.g. chronic atrial pacing).

In one embodiment, in the second, third and fourth aspects of the invention, the disease or condition is other than an atrial arrhythmia. Preferably, in these second and third aspects, the disease or condition is a cardiac (e.g. atrial) disease or condition, and/or the disease or condition is in a mammal such as a human.

Pharmaceutical Compositions (Formulations)

In order to use 5-HT₄ receptor antagonists, they will normally be formulated into a pharmaceutical composition in accordance with standard pharmaceutical practice.

5-HT₄ receptor antagonists (or “inhibitors”) may conveniently be administered by any of the routes conventionally used for drug administration, for instance, parenterally, orally, topically or by inhalation. 5-HT₄ receptor antagonists may be administered in conventional dosage forms prepared by combining it with standard pharmaceutically acceptable carriers according to conventional procedures. 5-HT₄ receptor antagonists may also be administered in conventional dosages in combination with a known, second therapeutically active compound. These procedures may involve mixing, granulating and compressing or dissolving the ingredients as appropriate to the desired preparation. It will be appreciated that the form and character of the pharmaceutically acceptable carrier is dictated by the amount of active ingredient with which it is to be combined, the route of administration and other well-known variables. The carrier(s) must be “acceptable” in the sense of being compatible with the other ingredients of the formulation and not deleterious to the recipient thereof.

The invention therefore also provides a pharmaceutical composition, for example for use in any of the methods/uses of the invention, comprising a 5-HT₄ receptor antagonist (e.g. comprising or being SB 207266 or a pharmaceutically acceptable salt thereof) in combination with a pharmaceutically acceptable carrier.

The pharmaceutically acceptable carrier employed may be, for example, either a solid or liquid. Exemplary of solid carriers are lactose, terra alba, sucrose, talc, gelatin, agar, pectin, acacia, magnesium stearate, stearic acid and the like. Exemplary of liquid carriers are syrup, peanut oil, olive oil, water and the like. Similarly, the carrier or diluent may include time delay material well known to the art, such as glyceryl mono-stearate or glyceryl distearate alone or with a wax.

A wide variety of pharmaceutical forms can be employed. Thus, if a solid carrier is used, the preparation can be tableted, placed in a hard gelatin capsule in powder or pellet form or in the form of a troche or lozenge. The amount of solid carrier will vary widely but preferably will be from about 25 mg to about 1 g. When a liquid carrier is used, the preparation will be in the form of a syrup, emulsion, soft gelatin capsule, sterile injectable liquid such as an ampoule or nonaqueous liquid suspension.

Two particularly preferred oral compositions for SB 207266, for human oral administration, are as follows:

| | | | |
|---|---|---|---|
| SB-207266 | 5.0 mg | SB-207266 | 5.0 mg |
| Microcrystalline | 30.0 mg | Microcrystalline cellulose | 50.0 mg |
| cellulose | | | |
| Mannitol | 112.0 mg | HPMC | 12.5 mg |
| Mg Stearate | 3.0 mg | Sodium Starch glycollate | 12.5 mg |
| | | Dicalcium phosphate | 167.5 mg |
| | | Mg stearate | 2.5 mg |
| Tablet weight | 150 mg | | 250 mg |

HPMC = hydroxypropylmethylcellulose

The dose in the second (right-hand) composition can readily be increased to 20 mg. The second composition is the result of a granulation process.
These and other suitable oral compositions for SB 207266 are described in Examples 4, 5, 6, 7 and 8 hereinbelow.
Dosage Regimens and Routes and Methods of Administration

5-HT₄ receptor antagonists (“inhibitors”) are preferably administered parenterally, that is by intravenous, intramuscular, subcutaneous, intranasal, intrarectal, intravaginal or intraperitoneal administration. The intravenous form of parenteral administration is generally preferred. Appropriate dosage forms for such administration may be prepared by conventional techniques.

5-HT₄ receptor antagonists (“inhibitors”) may also be administered orally. Appropriate dosage forms for such administration may be prepared by conventional techniques.

5-HT₄ receptor antagonists may also be administered by inhalation, that is by intranasal and oral inhalation administration. Appropriate dosage forms for such administration, such as aerosol formulations, may be prepared by conventional techniques.

5-HT₄ receptor antagonists may also be administered topically, that is by non-systemic administration. This includes the application of the 5-HT₄ receptor antagonist externally to the epidermis or the buccal cavity and the instillation of such a compound into the ear, eye and nose, such that the compound does not significantly enter the blood stream.

For all methods of use disclosed herein for 5-HT₄ receptor antagonists (“inhibitors”) such as SB 207266 or a pharmaceutically acceptable salt thereof, the daily oral dosage regimen will preferably be from about 0.1 to about 80 mg/kg of total body weight, preferably from about 0.2 to 30 mg/kg, more preferably from about 0.5 mg/kg to 15 mg/kg. The daily parenteral (e.g. intravenous) dosage regimen will preferably be from about 0.1 to about 80 mg/kg of total body weight, preferably from about 0.2 to about 30 mg/kg, and more preferably from about 0.5 mg/kg to 15 mg/kg. The daily topical dosage regimen will preferably be from 0.1 mg to 150 mg, administered one to four, preferably two or three times daily. The daily inhalation dosage regimen will preferably be from about 0.01 mg/kg to about 1 mg/kg per day.

Based on the above preferred dosage ranges and based on the in vivo results of in minipigs experiments shown in the Examples 1 and 2 hereinbelow, where doses of 0.3 and 1.0 mg/kg of SB-207266 administered intravenously were effective to treat atrial fibrillation and atrial remodelling, the following dosage ranges are preferred for prophylaxis or treatment of atrial arrhythmia (e.g. atrial fibrillation and/or atrial remodelling) comprising administering SB 207266 or a pharmaceutically acceptable salt thereof. The daily oral or parenteral (e.g. intravenous) dosage regimen will preferably be from about 0.1 mg/kg to 1.0 mg/kg of total body weight (e.g. 0.1 to 1.0 mg/kg), more preferably from about 0.2 mg/kg to 1.0 mg/kg (e.g. 0.2 to 1.0 mg/kg), still more preferably from 0.3 to 1.0 mg/kg, and most preferably from about 0.5 mg/kg to 1.0 mg/kg (e.g. 0.5 to 1.0 mg/kg), especially in a mammal such as a human. Alternatively, the daily oral or parenteral dosage regimen can be from about 0.2 mg/kg to about 0.5 mg/kg of total body weight, for example from about 0.2 mg/kg to 0.3 mg/kg of total body weight. For a human, for example weighing around 70-75 kg, a daily oral or parenteral (e.g. intravenous) dosage regimen of 0.3 to 1.0 mg/kg corresponds to approximately from (21-22.5) to (70-75) mg daily; about 0.2 mg/kg to 1.0 mg/kg corresponds to from (about 14-15) to (70-75) mg daily; about 0.5 mg/kg to 1.0 mg/kg corresponds to from (about 35-37.5) to (70-75) mg daily; about 0.2 mg/kg to about 0.5 mg/kg corresponds to from (about 14-15) to (about 35-37.5) mg daily; about 0.2 mg/kg to 0.3 mg/kg corresponds to from (about 14-15) to (21-22.5) mg daily.

Preferred daily doses for human oral or parenteral (e.g. intravenous) administration are:
a) 5-20 mg (e.g. as in the second specific oral composition of SB 207266 given above) and in particular 20 mg,
b) 50 mg
c) 80 mg.

Doses are measured as the weight of the SB 207266 free base, so that for salts of SB 207266 the weight of any acid(s) added to the free base to form the salt is excluded.

Therefore, a fifth aspect of the invention provides the use of N-[(1-ⁿbutyl-4-piperidinyl)methyl]-3,4-dihydro-2H-[1,3]oxazino[3,2-a]indole-10-carboxamide (SB 207266) or a pharmaceutically acceptable salt thereof in the manufacture of a medicament for the treatment or prophylaxis of atrial fibrillation in a mammal (e.g. human) by administering to the mammal a daily oral or parenteral dosage regimen of about 0.1 mg to 1.0 mg of the SB 207266 or salt thereof per kg of total body weight (measured as the free base).

This fifth aspect of the invention also provides a method of treatment or prophylaxis of atrial fibrillation in a mammal in need thereof, which comprises administering to said mammal a daily oral or parenteral dosage regimen of about 0.1 mg to 1.0 mg of N-[(1-ⁿbutyl-4-piperidinyl)methyl]-3,4-dihydro-2H-[1,3]oxazino[3,2-a]indole-10-carboxamide (SB 207266) or a pharmaceutically acceptable salt thereof per kg of total body weight (measured as the free base).

Also provided is N-[(1-ⁿbutyl-4-piperidinyl)methyl]-3,4-dihydro-2H-[1,3]oxazino[3,2-a]indole-10-carboxamide (SB 207266) or a pharmaceutically acceptable salt thereof for use in the treatment or prophylaxis of atrial fibrillation in a mammal (e.g. human) by administering to the mammal a daily oral or parenteral dosage regimen of about 0.1 mg to 1.0 mg of the SB 207266 or salt thereof per kg of total body weight (measured as the free base).

In all aspects (for example without limitation the first to the fifth aspects) of the invention, the daily oral or parenteral dosage regimen is preferably about 0.1 mg to 1.0 mg of SB 207266 or salt per kg of total body weight, more preferably about 0.2 mg/kg to 1.0 mg/kg, still more preferably from 0.3 to 1.0 mg/kg, for example about 0.5 mg/kg to 1.0 mg/kg, all measured as the free base. Alternatively or additionally, the daily oral or parenteral dosage regimen can be about 0.2 mg to about 0.5 mg, for example, about 0.2 mg to 0.3 mg, of the SB 207266 or salt thereof per kg of total body weight (measured as the free base).

More preferably, in all aspects of the invention, the daily dosage regimen comprises oral or parenteral (preferably oral) administration to a human of 20 mg, 50 mg or 80 mg of the SB 207266 or salt thereof (measured as the free base). These daily doses can be given as a single dose once daily, or can be given as two or more smaller doses at the same or different times of the day which in total give the specified daily dose.

A sixth aspect of the invention therefore provides the use of N-[(1-ⁿbutyl-4-piperidinyl)methyl]-3,4-dihydro-2H-[1,3]oxazino[3,2-a]indole-10-carboxamide (SB 207266) or a pharmaceutically acceptable salt thereof in the manufacture of a medicament for the treatment or prophylaxis of atrial fibrillation in a human by administering to the human a daily oral or parenteral (preferably oral) dosage of 20 mg, 50 mg or 80 mg of the SB 207266 or salt thereof (measured as the free base).

Also provided is a method of treatment or prophylaxis of atrial fibrillation in a human in need thereof, which comprises administering to said human a daily oral or parenteral dosage of 20 mg, 50 mg or 80 mg of the SB 207266 or salt thereof (measured as the free base).

Also provided is SB 207266 or a pharmaceutically acceptable salt thereof for use in the treatment or prophylaxis of atrial fibrillation in a human by administering to the human a daily oral or parenteral dosage of 20 mg, 50 mg or 80 mg of the SB 207266 or salt thereof (measured as the free base).

In all aspects of the invention, it is preferred that the 5-HT₄ receptor antagonist (e.g. SB 207266 or a pharmaceutically acceptable salt thereof) is used with/administered to patients with symptomatic atrial fibrillation (AF), and/or paroxysmal or persistent (preferably persistent) AF.

The 20, 50 and/or 80 mg human daily oral or parenteral doses and the about 0.2 mg/kg to 1.0 mg/kg daily doses are designed to minimise or reduce cardiovascular and/or other side-effects of administration of SB 207266. Preliminary studies indicate that human daily oral doses of about 120 mg or more of SB 207266 (corresponding to about 1.6 to 1.7 mg/kg/day or more in a mammal) might give rise to certain side-effects, and so preferably such high doses of SB 207266 should be avoided.

Therefore, it is preferable that the daily oral or parenteral dosage regimen is preferably less than about 1.5 mg of SB 207266 or salt per kg of total body weight, more preferably about 0.2 mg/kg to about 1.5 mg/kg, still more preferably from about 0.5 to about 1.5 mg/kg, for example about 1.0 mg/kg to about 1.5 mg/kg (e.g. 1.0 to 1.5 mg/kg or 1.0 to 1.3 mg/kg), all measured as the free base. Therefore, the invention also provides: (A) the use of SB 207266 or a pharmaceutically acceptable salt thereof in the manufacture of a medicament for the treatment or prophylaxis of atrial fibrillation in a mammal (e.g. human) by administering to the mammal a daily oral or parenteral dosage regimen of about 1.0 mg to about 1.5 mg (e.g. 1.0 to 1.5 mg or 1.0 to 1.3 mg) of the SB 207266 or salt thereof per kg of total body weight; (B) a method of treatment or prophylaxis of atrial fibrillation in a mammal in need thereof, which comprises administering to said mammal a daily oral or parenteral dosage regimen of about 1.0 mg to about 1.5 mg of SB 207266 or a pharmaceutically acceptable salt thereof per kg of total body weight; and/or (C) SB 207266 or a pharmaceutically acceptable salt thereof for use in the treatment or prophylaxis of atrial fibrillation in a mammal (e.g. human) by administering to the mammal a daily oral or parenteral dosage regimen of about 1.0 mg to about 1.5 mg of the SB 207266 or salt thereof per kg of total body weight; all weight being measured as the free base.

Preferably, in all aspects of the invention, the medicament/method of treatment or prophylaxis/5-HT₄ receptor antagonist (e.g. SB 207266 or a pharmaceutically acceptable salt thereof) is for use in the inhibition of symptomatic recurrences of atrial fibrillation in patients with paroxysmal or persistent AF (preferably persistent AF).

A preferred protocol for the inhibition of symptomatic recurrences of atrial fibrillation using SB 207266 in patients with persistent AF is described in detail in Example 3 hereinafter.

Loading Dose

With use/administration of SB 207266, it is usually desirable to achieve the full therapeutic response more promptly. In order to achieve this, it is believed that an initial larger “loading dose” (e.g. oral dose) of SB 207266 or a salt thereof can be employed to reach therapeutic concentrations more rapidly.

It has been found that steady-state plasma concentrations of SB 207266 are reached only after approximately 4 to 5 days of once daily dosing (the concentration on day 4 has been found to be about 90% of the steady-state plasma concentration). The elimination half-life T½ was found to be about 20-24 hours. It is believed that such a prolonged duration to achieve steady-state concentration is undesirable since patients with atrial fibrillation/remodelling who have been converted (cardioverted) to normal sinus rhythm following an episode of AF are more likely to have recurrences of AF soon after cardioversion. It is believed that the extent of accumulation following once daily oral dosing is approximately 1.5-fold. Therefore, the administration of approximately 1.5 times the daily dose (a “loading dose”) on Day 1 of administration of SB 207266 should result in attainment of pseudo steady-state plasma concentrations sooner. For example (without being bound by theory) 90% of the steady-state plasma concentration is thought to be achievable after about 24 hours or less of administering the loading dose, based in part on the modelling shown below. This should have a therapeutic benefit for an AF patient after cardioversion, e.g. by allowing earlier cardioversion after first dosing and/or by decreasing the chances of the patient reverting back to fibrillation soon after cardioversion and/or by decreasing hospitalisation time for the patient.

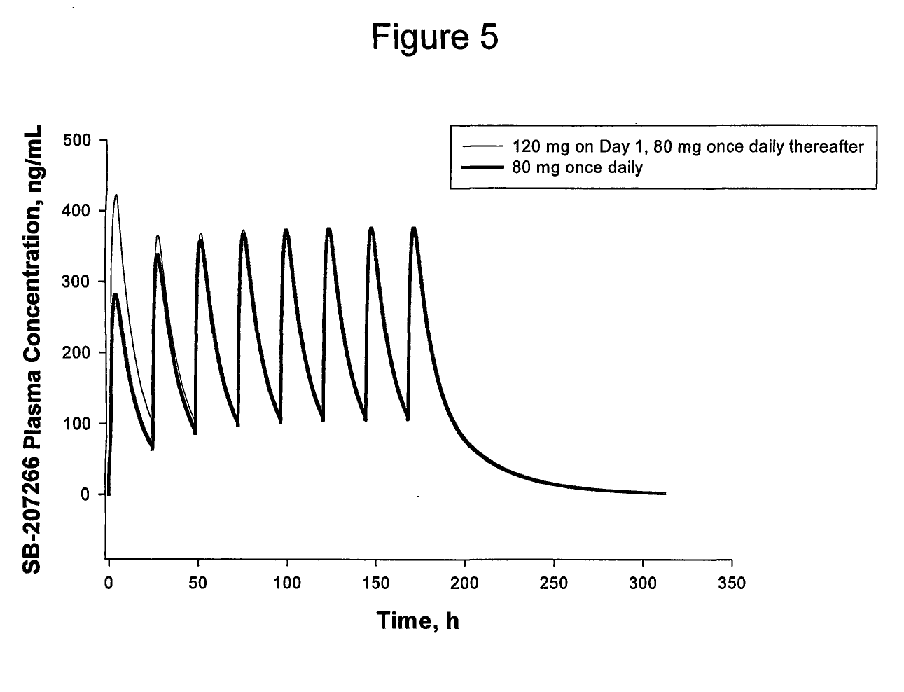

Preliminary population pharmacokinetic modelling has resulted in FIG. 5 which shows simulated SB-207266 plasma concentration vs time profiles for two regimens (120 mg on day 1 followed by 80 mg once daily for 7 days versus 80 mg once daily for 8 days). The simulations in FIG. 5 indicate that following a loading regimen of 1.5 times the maintainance dose, steady-state conditions are achieved more rapidly by 24 hours thereby reducing the telemetry monitoring period for each patient while still maintaining the maximum SB-207266 plasma concentrations within 10% of the target steady-state. The potential reduction of the telemetry monitoring period with a loading regimen would allow for an earlier patient discharge from an in-hospital treatment, with associated benefits in medical cost and convenience to the patient.

For these reasons, preferably SB 207266 or a pharmaceutically acceptable salt thereof is administered on the first day at a loading dose of about 1.2 to about 2.0 times (preferably about 1.25 to about 1.75 times, e.g. about 1.5 times) the daily maintainance dose and then is administered at the maintainance daily dose on subsequent days.

Therefore, in all aspects of the invention, the medicament, method or antagonist is for or employs administration of the SB 207266 or salt thereof on the first day at a loading dose of about 1.2 to about 2.0 times the daily maintainance dose, followed by administration of the SB 207266 or salt at the maintainance daily dose on subsequent days. Preferably, the loading dose is about 1.25 to about 1.75 times the daily maintainance dose, more preferably about 1.5 times the daily maintainance dose. Preferably, the daily maintenance dose comprises the daily oral or parenteral dosage or dosage regimen as defined in the fifth and/or sixth aspects of the invention.

Additionally, according to a seventh aspect of the invention there is provided the use of N-[(1-ⁿbutyl-4-piperidinyl)methyl]-3,4-dihydro-2H-[1,3]oxazino[3,2-a]indole-10-carboxamide (SB 207266) or a pharmaceutically acceptable salt thereof in the manufacture of a medicament for the prophylaxis or treatment of atrial arrhythmia (e.g. comprising atrial remodelling and/or atrial fibrillation) in a mammal (e.g. human) by administration of the SB 207266 or salt thereof on the first day at a loading dose of about 1.2 to about 2.0 times the daily maintainance dose, followed by administration of the SB 207266 or salt at the daily maintainance dose on subsequent days.

The seventh aspect of the invention also provides a method of treatment or prophylaxis of atrial arrhythmia (e.g. comprising atrial remodelling and/or atrial fibrillation) in a mammal (e.g. human) in need thereof, which comprises administering to said mammal an effective amount of N-[(1-ⁿbutyl-4-piperidinyl)methyl]-3,4-dihydro-2H-[1,3]oxazino[3,2-a]indole-10-carboxamide (SB 207266) or a pharmaceutically acceptable salt thereof, the method comprising administering the SB 207266 or salt thereof on the first day at a loading dose of about 1.2 to about 2.0 times the daily maintainance dose, and on subsequent days administering the SB 207266 or salt at the daily maintainance dose.

The seventh aspect of the invention also provides N-[(1-ⁿbutyl-4-piperidinyl)methyl]-3,4-dihydro-2H-[1,3]oxazino[3,2-a]indole-10-carboxamide (SB 207266) or a pharmaceutically acceptable salt thereof for use in the prophylaxis or treatment of atrial arrhythmia (e.g. comprising atrial remodelling and/or atrial fibrillation) in a mammal (e.g. human) by administration of the SB 207266 or salt thereof on the first day at a loading dose of about 1.2 to about 2.0 times the daily maintainance dose, followed by administration of the SB 207266 or salt at the daily maintainance dose on subsequent days.

Preferably, the loading dose is about 1.25 to about 1.75 times the daily maintainance dose, more preferably about 1.5 times (e.g. 1.5 times) the daily maintainance dose. Preferably, the daily maintenance dose comprises the daily oral or parenteral dosage or dosage regimen as defined in the fifth and/or sixth aspects of the invention.

Where the loading dose is 1.5 times the daily maintainance dose and where the mammal is a human, the loading dose is preferably 30 mg, 75 mg or 120 mg, and the daily maintainance dose is 20 mg, 50 mg or 80 mg respectively. The 20 mg, 50 mg or 80 mg doses/dosages can be according to the fifth and/or sixth aspects of the invention, so for example can be human daily oral or parenteral doses/dosages. See the Protocol in Example 3 hereinafter for an example of these doses and how they can be used.

Preferably, the loading dose is <1.6 to 1.7 mg/kg in a mammal (or in a human is <120 mg) of SB 207266 or salt thereof (measured as the free base), in order to minimise the risk of side-effects.

In all aspects of the invention, the daily maintenance dose can be given for a period clinically desirable in the patient, for example from 1 day up to several years (e.g. for the mammal's entire remaining life); for example from about (2 or 3 or 5 days, 1 or 2 weeks, or 1 month) upwards and/or for example up to about (5 years, 1 year, 6 months, 1 month, 1 week, or 3 or 5 days). Administration of the daily maintenance dose for about 3 to about 5 days or for about 1 week to about 1 year is typical.

Preferably, the loading dose is administered during an arrhythmic (e.g. atrial fibrillatory) episode in the mammal, and if the mammal is not in normal sinus rhythm after a period sufficient for the loading dose to take effect then the mammal is cardioverted back to normal sinus rhythm before administration of the maintenance dose. Therefore, preferably, the use, a method, a compound or an antagonist of the invention is for or employs administration of the loading dose during an arrhythmic (e.g. atrial fibrillatory) episode in the mammal, and which is for or employs administration of the maintenance dose after cardioversion of the mammal back to normal sinus rhythm in the event that the mammal is not in normal sinus rhythm after a period sufficient for the loading dose to take effect. More details follow.

Preferred Administration Methods Including Optional Cardioversion

An eighth aspect of the invention provides a method of treating a mammal who is experiencing an arrhythmic (e.g. atrial fibrillatory) episode, comprising:
(a) administering N-[(1-ⁿbutyl-4-piperidinyl)methyl]-3,4-dihydro-2H-[1,3]oxazino[3,2-a]indole-10-carboxamide (SB 207266) or a pharmaceutically acceptable salt thereof at a dosage or dosage regimen according to the fifth and/or sixth aspects of the invention and/or at a loading dose as defmed in the seventh aspect of the invention,
(b) waiting for a period sufficient for the dose, dosage or dosage regimen in step (a) to take effect at least partially,
(c) optionally measuring whether the mammal has reverted to normal sinus rhythm,
(d) cardioverting the mammal back to normal sinus rhythm in the event that the mammal is not in normal sinus rhythm after the period in step (b), and then
(e) optionally administering as necessary a further dose of the SB 207266 or salt thereof.

Preferably, in steps (a) and/or (e), oral administration of the SB 207266 or salt thereof is used.

Preferably, the period in step (b) is about 0.25 to about 8 hours, more preferably about 0.5 to about 4 hours, more preferably about 1 to about 4 hours, still more preferably about 1 to about 3 hours, e.g. about 2 hours. This is particularly preferred for oral administration in step (a). The maximum plasma concentration (Cₘₐₓ) has been found to be about 2 hours after oral administration of SB 207266.

In step (d), the cardioversion can comprise pharmacological and/or DC cardioversion; preferably, in step (d) DC cardioversion is used.

Preferably, step (a) comprises administering the SB 207266 or salt thereof at a loading dose according to the seventh aspect of the invention, and step (e) comprises optionally administering as necessary the SB 207266 or salt thereof at the daily maintenance dose on subsequent days according to the seventh aspect of the invention.

Preferably, step (e) comprises optionally administering as necessary the SB 207266 or salt thereof at a dosage or dosage regimen according to the fifth and/or sixth aspects of the invention.

A plurality of doses of the SB 207266 or salt thereof can optionally be administered in step (e) over a period, as necessary.

Preferably, the method according to the eigth aspect of the invention comprises administration of anticoagulation therapy (e.g. comprising administration of warfarin) to the mammal before, during and/or after the period during which the method of treatment according to the eighth aspect of the invention takes place.

In all aspects of the invention, the mammal should preferably receive anticoagulation therapy (e.g. comprising warfarin administration) throughout some (e.g. most) or all of the period during which the 5-HT4 antagonist (e.g. SB 207266 or salt thereof) is administered. Therefore, in all aspects of the invention, the use/method/antagonist/compound is preferably for co-administration of the antagonist and anticoagulation therapy (e.g. comprising administration of warfarin) to the mammal.

All publications, including but not limited to patents and patent applications, cited in this specification are herein incorporated by reference as if each individual publication were specifically and individually indicated to be incorporated by reference herein as though fully set forth.

The invention will now be described by reference to the following Examples which are merely illustrative and are not to be construed as a limitation of the scope of the present invention. Some of the Examples are illustrated by the Figures in which:

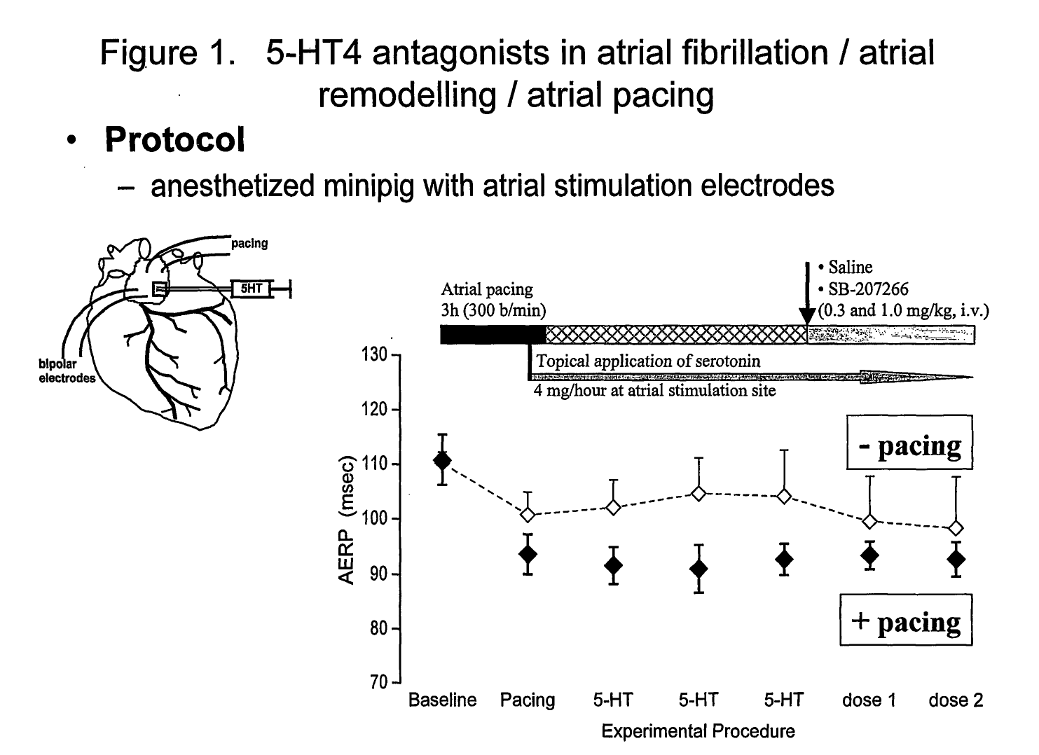

FIG. 1, entitled “5-HT4 antagonists in atrial fibrillation/atrial remodelling/atrial pacing; Protocol—anesthetized minipig with atrial stimulation electrodes”, shows a schematic outline of the protocol used in the experimental generation of 5-HT-induced atrial fibrillation and atrial remodelling in minipig, and its treatment with a 5-HT4 antagonist (SB 207266), according to Examples 1 and 2;

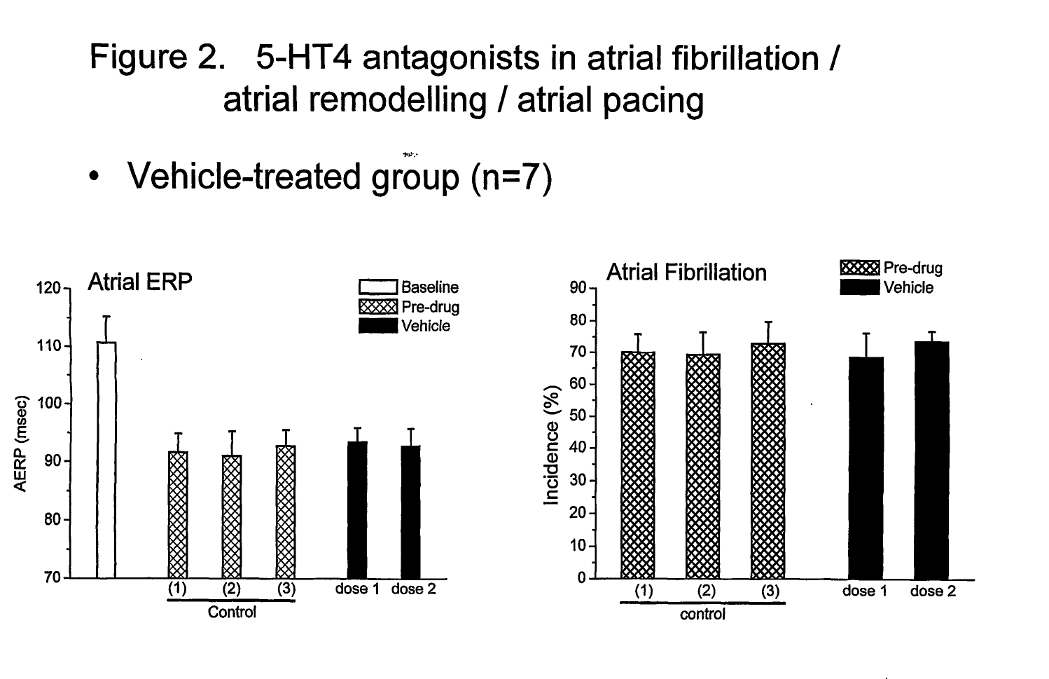

FIG. 2, entitled “5-HT4 antagonists in atrial fibrillation/atrial remodelling/atrial pacing; Vehicle-treated group (n=7)”, shows the changes in atrial ERP and incidence of atrial fibrillation induced/caused by rapid atrial pacing and 5-HT, in a vehicle-treated group of 7 minipigs;

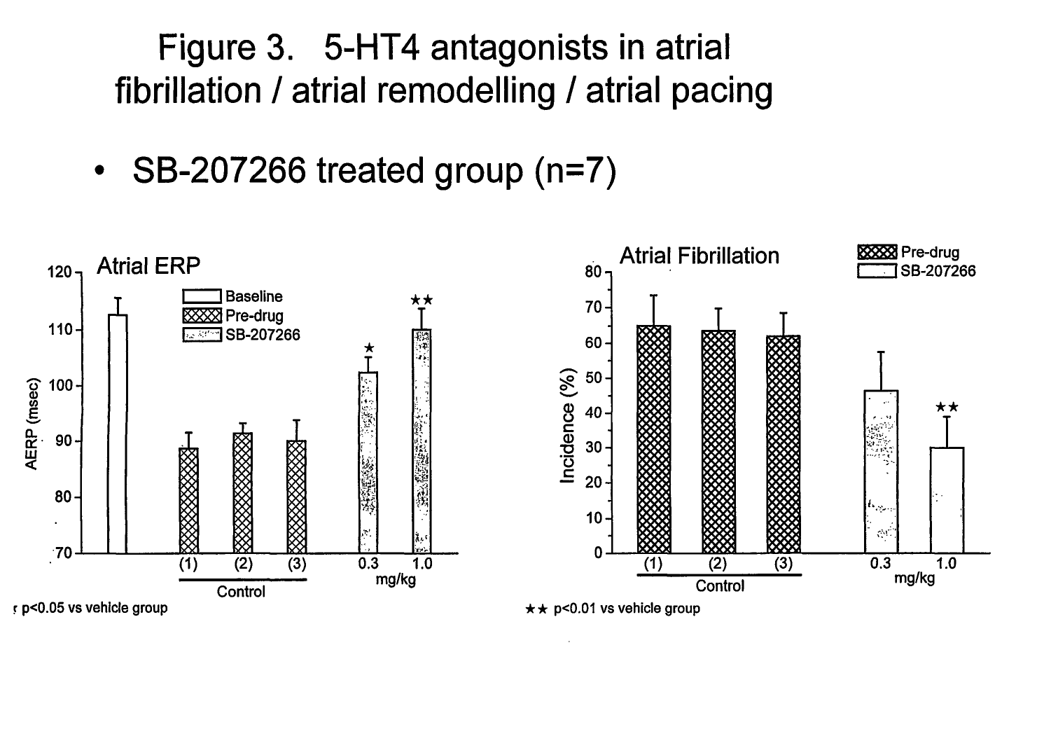

FIG. 3, entitled “5-HT4 antagonists in atrial fibrillation/atrial remodelling/atrial pacing; SB-207266 treated group (n=7)”, shows the changes in atrial ERP and incidence of atrial fibrillation induced/caused by rapid atrial pacing and 5-HT, in a group of 7 minipigs treated with SB-207266;

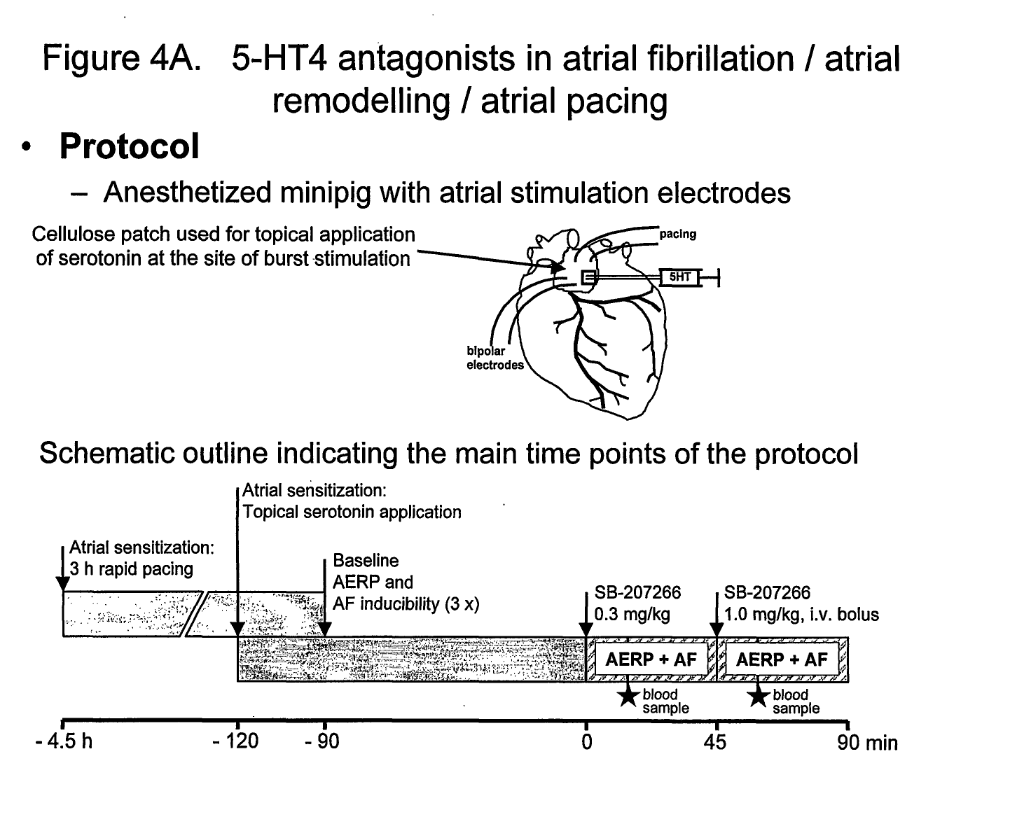

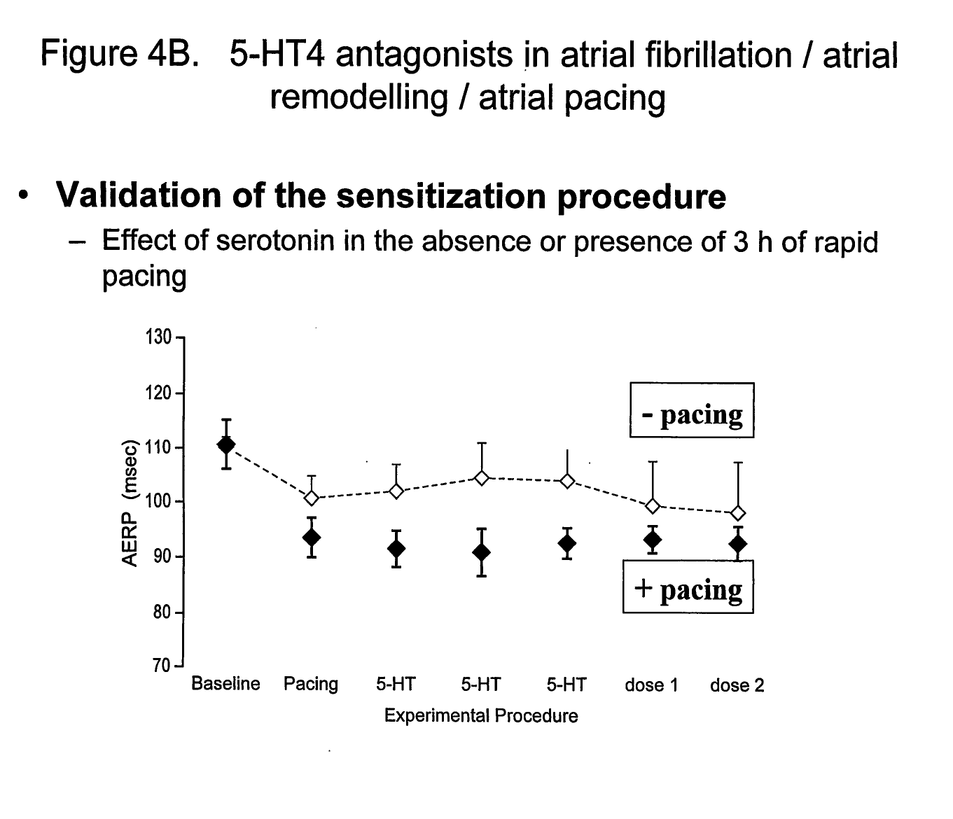

FIG. 4A is a differently presented version of the FIG. 1, and is a schematic outline indicating the main time points of the protocol used in 5-HT-induced atrial fibrillation and atrial remodelling in minipig, as described in Example 2;

FIG. 4B is a graph showing the effect of serotonin (5-HT) on the AERP, in the absence or presence of 3 hours of rapid atrial pacing and in the absence of SB 207266, when using the FIG. 4A minipig protocol; and

FIG. 5 shows simulated SB-207266 plasma concentration vs time profiles for two regimens (120 mg on day 1 followed by 80 mg once daily for 7 days versus 80 mg once daily for 8 days).

### EXAMPLES

SB 207266 -N-[(1-ⁿbutyl-4-piperidinyl)methyl]-3,4-dihydro-2H-[1,3]oxazino[3,2-a]indole-10-carboxamide—is made using the synthetic methods decribed in the introduction, i.e. as described in one or more of WO 93/18036; WO 98/07728, WO 98/11067; WO 00/03983; and WO 00/03984.

### Example 1

Experimental Atrial Fibrillation/Atrial Remodelling Test Results with SB-207266

The antiarrhythmic efficacy of SB-207266 (0.3 and 1.0 mg/kg, intravenous) was evaluated on the inducibility of AF in a model of 5-HT-induced atrial arrhythmia in anesthetized Yucatan minipigs. As shown in FIG. 1, prior to AF induction, animals were sensitized by 3 hours of rapid atrial pacing (200 msec cycle length) and concomitant topical application of 5-HT (4 mg/h) at the atrial stimulation site. The atrial effective refractory period (AERP) and AF inducibility were determined during programmed stimulation and burst electrical pacing.

In both vehicle- and drug-treated groups, rapid atrial pacing and application of 5-HT caused a reduction of AERP from 111.6±2.6 to 90.9±2.1 msec, before application of the vehicle or drug—see black diamonds (♦) in FIG. 1 and left-hand bar graph in FIG. 2. A smaller reduction of AERP was seen when 5-HT was added without atrial pacing—see white diamonds (⋄) in FIG. 1. As shown in the right hand bar graphs in FIGS. 2 and 3, the pre-drug AF inducibility (% incidence of AF) caused by 10 successive bursts of pacing (2 sec-burst at 20 msec cycle length) was stable and reproducible (76±8, 69±7, 73±4%, n=7 in vehicle group, FIG. 2).

As shown in FIG. 3, application of SB-207266 after the atrial pacing and application of 5-HT caused a dose-dependent increase in AERP from 90.0±2.7 to 102.3±2.7 and 110.0±3.6 msec, respectively at 0.3 and 1.0 mg/kg (p<0.01 vs vehicle). At the same time, AF inducibility was reduced from 64±6% in the absence of drug to 46±11 and 30±9%, respectively at 0.3 and 1.0 mg/kg (p<0.01).

These results suggest that SB-207266 has effective properties in the prophylaxis or treatment of atrial remodelling (or AF) caused by rapid atrial pacing, associated with selective prolongation of atrial refractoriness (lengthening of the AERP).

### Example 2

More Detailed Experimental Atrial Fibrillation/Atrial Remodelling Test Results with SB-207266

The following is a more detailed description of the experimental procedures, results, discussion and conclusions given in Example 1 above. Reference is again made to the FIGS. 1-3, and the additional FIGS. 4A and 4B.

Example 2

Materials and Test Systems

Materials. The following technical equipment was used to carry out this study:
Anesthesia vaporizer: Boyle International 2, Medishield, Harlow, England.
Artificial repiration pump: Model 613, Harvard, South Natick, Mass., USA.
Heating pad water pump: Model TP-420, Gaymar Industries, NY, USA.
Blood gas analyzer: ABL 500, Radiometer, Copenhagen NV., Denmark
Pressure transducer: Model P23 ID, Gould Electronics, Cleveland, Ohio, USA.
Drug infusion: B Braun Melsungen AG, Germany
Electrophysiological stimulation: S8800 stimulator and SIU-5 stimulation isolation unit, Grass Instrument Co., Quincy, Mass., USA.
Chart paper recorder: TA-5000 polyrecorder, Gould Electronics.
Digital tape recorder: DTR 1800 Biologic, Claix, France.

Animals. Male Yucatan minipigs (12-17 kg weight) were obtained from Charles River (Saint-Aubin les Elboeuf, France) and were maintained at rest over a 2-week acclimatision period prior to experiments.

Surgical Preparation of Animals. The minipigs (Charles River, France) were fasted and premedicated (2 mg/kg diazepam+15 mg/kg ketamine, i.m.) before induction of anesthesia by isoflurane inhalation (5% for induction followed by 0.5 to 1.5% for technical preparation) in a mixture of 25% O₂ and 75% N₂O. The long term anesthesia was maintained with an i.v. infusion of sodium pentobarbital (12 mg/kg/h). A mechanical ventilator (Harvard pump 613) was used to provide artificial respiration during the left thoracotomy in order to keep arterial blood gases and pH within the normal limits (ABL 500 analyzer). Fluid filled catheters were placed in the femoral artery and vein to measure the arterial pressure (P23 ID transducer) and for drug administrations, respectively. Leads II, III and a precordial lead of electrocardiogram were placed for monitoring of standard ECG parameters. Two pairs of electrodes were hooked to the left atrial wall for subsequent stimulation (S8800 stimulator and SIU-5 unit) and for measurement of an atrial electrogram.

### Example 2

Experimental Procedures

Sensitization of the Atrial Tissue. The left atrial appendage was sensitized by rapid atrial pacing (200 ms cycle length over 3 hours) to generate an initial electrical remodelling of the tissue [A. Goette et al., 1996, Circulation, 94, 2968-2974]. Then, a solution of 5-HT (serotonin) (4 mg/h, starting 30 min before end of atrial pacing) was applied locally using a cellulose patch placed close to the stimulating electrodes, and this 5-HT application was maintained until the end of the experiment. The cellulose patch retains the 5-HT in contact with the tissue. After this period of sensitization and under constant topical application of 5-HT, baseline atrial refractoriness and inducibility of AF were determined (see the cross-hatched area in FIG. 1, cross-hatched “pre-drug” bar graphs in FIGS. 2 and 3, and shaded box from −90 mins to 0 mins in FIG. 4A).

Electrophysiologic Study. The atrial effective refractory period (AERP) was determined using a conventional single extrastimulus technique as previously described [A. Bril, B. Gout et al., J. Pharmacol. Exp. Ther., 1996, 276, 637-646.]. Briefly, a 8-stimulus train at a basic cycle length 20% shorter than the sinus rhythm was followed by a single premature extrastimulus (4 ms, 1.5 times threshold current) introduced at progressively shorter coupling intervals from the atrial pacing until no atrial response was obtained. The AERP represents the longest coupling interval which failed to induce a propagated response in the tissue.

After determination of AERP, the atrial fibrillation (AF) challenge was started. AF was induced following atrial pacing by a 2 sec-burst of stimulation (basic cycle length 20 ms cycle length, 2 ms duration, twice the diastolic threshold) introduced within the vulnerable window (AERP+10 ms). AF was defined as at least 1 sec of irregular electrical activity measured on atrial electrogram.

Study Design and Dosing. After assessment of reproducible baseline responses of animals (3 successive AF challenges), the minipigs were randomly assigned to receive either sterile distilled water (vehicle group, n=7), or escalating doses of SB-207266 (0.3 and 1.0 mg/kg, n=7) given i.v. over a 10 min period 15 min before determination of AERP and subsequent AF challenge. SB-207266 was dissolved in sterile distilled water and adequate drug solutions were prepared every day. Treatment with vehicle represented a volume of distilled water similar to that used for drug solution (10 ml). Each dose of SB-207266 was administered at 45 min intervals to allow recovery of animals from previous burst pacing challenge. Brief schemes indicating the main time points of the protocol are presented in FIGS. 1 and 4A.

Evaluation of Plasma Concentration of SB-207266. For each AF challenge, blood samples were collected on EDTA (6%) 5 min after the end of the bolus administration of drug (time point 15 min, see asterisks in FIG. 4A) and centrifuged (1500×g, 10 min at 4° C.). The plasma samples were stored at −80° C. for subsequent analysis. Plasma samples were not collected in the vehicle group. The determination of plasma concentration of SB-207266 was performed by LC/MS/MS with a LLQ for this assay of 5 ng/ml in the pig.

### Example 2

Data Handling & Analysis

Measurements and Calculations. All parameters were monitored on a chart paper polyrecorder (TA-5000) and digital recording was performed throughout the protocol (DTR 1800). When measured, heart rate was calculated from the ECG and the mean arterial blood pressure was calculated from the pulse pressure. A corrected QT was determined according to the Bazett's formula (QTc=QT (ms)/RR (sec)^{1/2}). AF inducibility was expressed as a percentage of responses obtained from 10 successive bursts and the mean duration of AF episodes recorded during the sequence of 10 burst stimulations was expressed in seconds.

Statistical Analysis. Values are expressed as mean±SEM. Comparisons were performed using analysis of variance (ANOVA) followed by a Newman-Keuls test for multiple pairwise comparisons. The drug effects were measured using ANOVA for repeated measures. The inducibility of AF in response to burst stimulation was analysed using a Kruskal-Wallis rank sum test. All statistics were performed using the Statistica 5.1 release Package (StatSoft, Inc., Tulsa, Okla., USA).

### Example 2

Results

5-HT-Induced Atrial Fibrillation in Minipig. In this model, application of rapid atrial pacing during 3 hours prior to topical application of 5-HT was necessary to generate enough electrical remodelling in the atrial tissue to favour the occurrence of AF in response to burst stimulation. For this purpose, the effects of various interventions, including rapid atrial pacing alone, 5-HT alone and the combination of both pacing and 5-HT were studied on the changes in AERP. After 3 hours of rapid atrial pacing (200 ms basic cycle length) the AERP was significantly decreased from 110.7±4.6 ms to 93.6±3.6 ms (n=7; p<0.01). 5-HT given alone as a topical application for 3 hours did not significantly change the AERP (104.0±6.5 ms versus 110.2±1.9 ms in control, n=6). The application of 5-HT after rapid atrial pacing did not cause a significant further reduction in AERP which was 91.8±3.3 ms (compared to 93.6±3.6 ms with pacing alone). See the graphs in FIGS. 1 and 4B, and the left-hand bar graph in FIG. 2.

As shown in the right-hand bar graph in FIG. 2, in pigs subjected to rapid right atrial pacing and simultaneous application of 5-HT, successive AF challenges performed in the vehicle-treated group exhibited a stable and reproducible inducibility of AF (71±5% of positive response from ten bursts of pacing, with a range 69 to 74% over 5 challenges). The average duration of AF episodes measured in response to burst pacing was 2.5±0.5 sec (range 1.2 to 6.7 sec) and was stable during the successive AF challenges, as shown in Table 1 below.

Effect of SB-207266 on 5-HT-Induced AF. Intravenous administration of increasing doses of SB-207266 (0.3 and 1.0 mg/kg) in sensitized pigs induced a dose-dependent increase in AERP from a predrug value of 90±3 ms to 102±3 and 110±4 ms at 0.3 and 1.0 mg/kg, respectively (p<0.01 vs vehicle). The decrease in AERP caused by rapid atrial pacing plus 5-HT application was fully restored after administration of 1.0 mg/kg SB-207266 (see FIG. 3, left-hand bar graph). At the same time, a dose-related reduction of AF inducibility was observed, from 64±6% prior to treatment to 46±11% at 0.3 mg/kg (p=0.139 vs vehicle) and 30±9% at 1.0 mg/kg (p<0.01) (see FIG. 3, right-hand bar graph). The mean duration of AF episodes was slightly but significantly reduced from 1.9±0.4 sec prior to drug treatment to 1.1±0.4 sec after administration of 0.3 mg/kg SB-207266 (p<0.05 vs vehicle). No further reduction of the mean duration of AF was observed at higher dose, as shown in Table 1 below.

Plasma concentrations of SB-207266. The circulating concentration of SB-207266 was measured in plasma samples collected 5 min after the end of each bolus dose of SB-207266. The plasma level of SB-207266 observed with 0.3 mg/kg SB-207266 was 137.7±15.2 ng/ml (n=6) and reached 562.3±40.1 ng/ml at 1.0 mg/kg (n=5).

TABLE 1
Mean Duration of AF Episodes caused by Rapid
atrial Pacing and 5-HT in Minipig
Comparison between vehicle-treated and
SB-207266-treated animals

| | vehicle (10 ml bolus) | | | SB-207266 (mg/kg, i.v.) | | |
|---|---|---|---|---|---|---|
| | | ve- | | Pre- | | |
| | Control | hicle | vehicle | drug | 0.3 | 1.0 |
| Duration of | 2.5 ± 0.5 | 2.9 ± | 2.1 ± | 1.9 ± | 1.1 ± 0.2 | 1.1 ± 0.4 |
| AF (sec) | | 0.8 | 0.3 | 0.4 | | |
| P value vs: | | | | | | |
| vehicle | | | | NS | P < 0.05 | P < 0.05 |
| Pre-drug | | NS | NS | | P < 0.05 | P < 0.05 |

NS: not significant

### Example 2

Discussion

The results of the present study show that 5-HT exhibits a minimal effect on AERP compared to rapid atrial pacing. In the goat it has been shown that at the time of rapid atrial pacing initiation AERP decreased after 6 hours (physiological rate adaptation) and that this reduction was further observed with time [M. C. E. F. Wijffels et al., Circulation, 1997, 96, 3710-3720]. Our results show that 3 hours of rapid atrial pacing in minipigs is sufficient to obtain a stable reduction of AERP which characterises the electrical remodelling. In addition our results show that the application of 5-HT either alone or in the presence of rapid atrial pacing marginally changed the AERP. This suggests that 5-HT may not be directly involved in the electrophysiological mechanisms leading to AF but might rather play a facilitating role in AF.

Although 5-HT induced a minimal effect on AERP and AF incidence, the i.v. administration of the 5-HT₄ receptor antagonist, SB-207266, prevents/inhibits (or reverses) the AERP reduction and protects from AF inducibility in a dose-dependent manner. These results are shown to be related to the plasma concentration of the drug. These results suggest that inhibition (antagonism) of the 5-HT₄ receptor, for example by administering SB-207266, appears to lead to atrial antifibrillatory effects.

### Example 2

Conclusions

SB-207266 was shown to reverse significantly the reduction of AERP caused by combined rapid atrial pacing and topical application of 5-HT and to reduce significantly the incidence of AF episodes. These results suggest that SB-207266 and 5HT₄ receptor antagonists in general may be effective at reducing/treating atrial fibrillation, associated with a restoration (increase) of the atrial ERP characterizing a reversal of the atrial electrical remodelling observed in the presence of 5-HT and atrial pacing.

The results with SB-207266 described in Example 2 (and also in Example 1), appear to illustrate a novel approach for the treatment or prophylaxis of atrial remodelling and/or atrial arrhythmias such as atrial fibrillation, by administration/use of a 5-HT₄ receptor antagonist such as any of the compounds described herein.

### Example 3

Protocol for the Treatment or Prophylaxis of Atrial Fibrillation and/or Atrial Remodelling in Humans Using Orally Administered SB 207266

A currently preferred protocol for the treatment or prophylaxis of atrial remodelling and/or atrial fibrillation using SB 207266 or a salt thereof is now described in detail.

This Protocol describes administration of SB 207266 or the salt (hereinafter “SB 207266”) to patients with symptomatic persistent atrial fibrillation (AF). The objective is the inhibition of symptomatic recurrences of atrial fibrillation in these patients with persistent AF. Patients with symptomatic persistent AF, of duration ≧48 hrs and <6 months, who require cardioversion (e.g. DC cardioversion) are suitable. Symptoms of persistent AF may for example include palpitations, etc. Patients preferably either have:
therapeutic anticoagulation (e.g. warfarin) for ≧3 weeks before commencement of treatment, or
in the absence of therapeutic anticoagulation for ≧3 weeks, they have a transesophageal echocardiography (TEE) which is negative for clot and have received intravenous heparin until aPTT is stable and in the therapeutic range.

Patients receive SB 207266 preferably after such therapeutic anticoagulation, or after TEE in addition to iv heparin.

SB 207266 (e.g. as free base, but more preferably as the hydrochloride salt SB 207266-A) is generally administered at daily oral doses of 20 mg, 50 mg or 80 mg uid (measured as the free base). However, on day 1 of the administration of SB 207266, it is generally administered at a single oral loading dose of 1.5 times (1.5×) the dosage allocated for the daily maintenance therapy. Therefore, preferably, a single oral loading dose of 30 mg, 75 mg or 120 mg is given on day 1, followed by a daily dose of 20 mg, 50 mg or 80 mg respectively on subsequent days.

About two hours after administration of the first-day 1.5× oral loading dose of SB 207266, patients remaining in atrial fibrillation (and/or not pharmacologically cardioverted) preferably then undergo direct current (DC) cardioversion. Either of the following mono or bi-phasic cardioversion algorithms can be followed.

| Shock sequence | Mono-phasic | Bi-Phasic |
|---|---|---|
| 1st Shock | 200 Joules | 170 Joules |
| 2nd Shock | 250 Joules | 200 Joules |
| 3rd Shock | 300 Joules | 230 Joules |

If the patient does not revert to normal sinus rhythmn (NSR) after the 3rd shock using one of the above sequences the doctor may at his discretion proceed with further attempts at different energies. Successful cardioversion is defined as maintenance of NSR for ≧1 hr.

Following a successful DC cardioversion to NSR, administration of SB 207266 to the patient can be continued once daily for 6 months (for example), or for shorter or longer periods. Those patients who spontaneously revert to normal sinus rhythmn (NSR) can also receive SB 207266 once daily for (e.g.) 6 months. Patients who experience a recurrence of AF during this daily treatment can be DC cardioverted back to sinus rhythm and can continue to receive SB 207266.

Patients should preferably continue on anticoagulation therapy (e.g. warfarin) throughout the period during which SB 207266 is administered.

The most preferred Protocol is therefore given below:

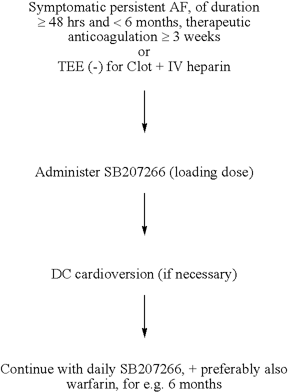

A “symptomatic recurrence” of AF includes or means an episode of palpitations or other symptoms typical for the patient. This can be further established by either a ECG (e.g. 12-lead ECG) recording showing evidence of atrial fibrillation or a rhythm strip recorded on a event recorder device and optionally reviewed by the doctor.

### Examples 4, 5, 6, 7 and 8

SB 207266 Pharmaceutical Compositions

### Example 4

A preferred oral composition for SB 207266, for human oral administration, is as follows:

| | |
|---|---|
| SB-207266 | 5.0 mg |
| Microcrystalline cellulose | 30.0 mg |
| Mannitol | 112.0 mg |
| Mg Stearate | 3.0 mg |
| Tablet weight | 150 mg |

### Example 5

A more preferred oral composition for SB 207266, for human oral administration, is as follows:

| | |
|---|---|
| SB-207266 | 5.0 mg |
| Microcrystalline cellulose | 50.0 mg |
| HPMC (hydroxypropylmethylcellulose) | 12.5 mg |
| Sodium Starch glycollate | 12.5 mg |
| Dicalcium phosphate | 167.5 mg |
| Mg stearate | 2.5 mg |
| Tablet weight | 250 mg |

The dose in this composition can readily be increased to 20 mg. This composition is the result of a granulation process.

### Example 6

The tablet of Example 5 can be varied by increasing the dose of SB 207266 from 5 mg to up to 20, 60 or 80 mg (measured as the free base), and by decreasing the amount of dicalcium phosphate accordingly while keeping the 250 mg tablet weight constant.

### Example 7

SB-207266-A Tablets with 10, 25, and 40 mg Strength (Measured as Pure Free Base)

Tablets containing the hydrochloride salt of SB 207266 (SB 207266-A) in amounts of 10, 25 or 40 mg (measured as the free base) were made according to the composition in the table below. These tablets were designed to be used in the treatment Protocol described in Example 3, using two of the tablets a day for a total daily dose of 20, 50 and 80 mg respectively as called for in the Protocol.

| | | Quantity (mg/tablet) | | |
|---|---|---|---|---|
| | | 10 mg | 25 mg | 40 mg |
| | | tablet | tablet | tablet |
| Ingredient | Function | strength | strength | strength |
| Active Ingredient | | | | |
| SB-207266-A | API | 11.0* | 27.5* | 44.0* |
| Other Ingredients | | | | |
| Microcrystalline Cellulose | Compression | 50.0 | 50.0 | 50.0 |
| (e.g. Ph. Eur. or NF) | & | | | |
| | granulation | | | |
| | aid | | | |
| Hydroxypropylmethyl | Binder | 12.5 | 12.5 | 12.5 |
| cellulose (e.g. USP) | | | | |
| Sodium starch glycollate (e.g. | Disintegrant | 12.5 | 12.5 | 12.5 |
| NF or Ph Eur) | | | | |
| Calcium hydrogen phosphate | Major | 161.5 | 145.0 | 128.5 |
| dihydrate | diluent | | | |
| (Dibasic Calcium Phosphate | | | | |
| dihydrate) (e.g. Ph. Eur. or | | | | |
| USP) | | | | |
| Magnesium Stearate (e.g. Ph. | Lubricant | 2.5 | 2.5 | 2.5 |
| Eur. or NF) | | | | |
| Purified Water** (e.g. Ph. | Granulating | ** | ** | ** |
| Eur. or USP) | solvent | | | |
| Opadry White YS-1-7003 | Film Coat | 6.25 | 6.25 | 6.25 |
| Purified Water** | | ** | ** | ** |
| Total Tablet Weight | | 256.25 | 256.25 | 256.25 |

*Equivalent to 10, 25, 40 mg respectively of pure free base
**Removed during processing

The SB-207266-A tablets of Example 7 are packed into high density polyethylene (HDPE) bottles with plastic, child-resistant, induction seal caps.

The formulation used a wet granulation process using an insoluble major excipient, Dibasic calcium Phosphate dihydrate (or Dicalcium phosphate). Dibasic calcium Phosphate dihydrate is the major diluent together with microcrystalline cellulose which is added to disperse the granulating solvent and to aid in the overall compressibility. The binding agent added is hydroxypropylmethyl cellulose and the granulation is carried out in a conventional mixer granulator. The granule mix is dried, screened and mixed with sodium starch glycollate as a disintegrant and magnesium stearate as a lubricant to form the compression mix. Tablets are produced on a suitable rotary tablet press, and can be either oval or round in shape.

### Example 7

Detailed Manufacturing Process, In-process Controls, and Assembly Process

SB-207266-A, microcrystalline cellulose, dibasic calcium phosphate dihydrate, and hydroxypropylmethyl cellulose are blended together. Purified water is added to the blended powders while mixing in a high shear mixer-granulator. The granules are dried in a fluid bed drier and are then transferred to a mixer, where they are blended with sodium starch glycollate and magnesium stearate. The lubricated mix is compressed into tablet cores using a rotary tablet press. The tablet cores are film coated using an aqueous dispersion of Opadry White YS-1-7003.

Procedure:

1.0 Granulation.
   1.1 Blend the SB-207266, microcrystalline cellulose, hydroxypropylmethyl cellulose and dibasic calcium phosphate dihydrate in a suitable high shear mixer-granulator.
   1.2 Add the purified water to effect the the granulation.
   1.3 Dry the granules in a fluid bed drier.
   1.4 Pass the dried granules through a stainless steel screen using a suitable mill.
   1.5 Determine the yield of the granules.
2.0 Manufacture of Compression Mix.
   2.1 Blend the required quantites of sodium starch glycollate and magnesium stearate with the dried granules
   2.2 Determine the yield of compression mix.
3.0 Tablet Compression.
   3.1 Transfer the compression mix to a suitable tablet machine.
   3.2 Compress the tablets.
   3.3 Determine the yield of the compressed tablets.
4.0 Film Coating.
   4.1 Transfer the tablet cores to a suitable coating machine.
   4.2 Rotate the cores and spray on aqueous dispersion of Opadry.
   4.3 Release test samples are taken randomly from the batch and appropriately labelled.
5.0 Bottle filling
   5.1 HDPE bottles are filled to the appropriate fill count, induction sealed and fitted with a child resistant cap using suitably automated equipment.

### Example 8

In a modification of Example 7, formulations containing 20 mg, 50 mg and 80 mg SB-207266 (as the hydrochloride salt, but the dose given being measured as the free base) can been used to make tablets, maintaining the total tablet weight of 256.25 mg and the other excipient amounts in the Example 7 compositions, but adjusting the amount of Dibasic Calcium Phosphate dihydrate used as the amount of SB 207266 varies. These tablets can be round or oval.

Tests for 5-HT₄ Receptor Antagonist Activity and Activity of SB 207266

1) Guinea Pig Colon

This animal model is described by Wardle K A and Sanger G J (1993), Br J Pharmacol; 110 1593-1599.

Male guinea-pigs, weighing 250-400 g are used. Longitudinal muscle-myenteric plexus preparations, approximately 3 cm long, are obtained from the distal colon region. These are suspended under a 0.5 g load in isolated tissue baths containing Krebs solution bubbled with 5% CO₂ in O₂ and maintained at 37° C. In all experiments, the Krebs solution also contains methiothepin 10⁻⁷M and granisetron 10⁻⁶M to block effects at 5-HT₁, 5-HT₂ and 5-HT₃ receptors.

After construction of a simple concentration-response curve with 5-HT, using 30 s contact times and a 15 min dosing cycle, a concentration of 5-HT is selected so as to obtain a contraction of the muscle approximately 40-70% maximum (10⁻⁹M approx). The tissue is then dosed every 15 min with this concentration of 5-HT. In some experiments, this tissue was dosed alternately with an approximately equi-effective concentration of the nicotine receptor stimulant, dimethylphenylpiperazinium (DMPP). After obtaining consistent responses to 5-HT (and when appropriate, DMPP), increasing concentrations of a putative 5-HT₄ receptor antagonist are then added to the bathing solution. The effects of this compound are then determined as a percentage reduction of the contractions evoked by 5-HT or by DMPP. From this data, pIC₅₀ values are determined, being defined as the −log concentration of antagonist which reduces the contraction by 50%. A compound which reduces the response to 5-HT but not to DMPP is believed to act as a 5-HT₄ receptor antagonist.

SB 207266 had a particularly good activity.

In the presence of 5-HT₁, 5-HT₂ and 5-HT₃ receptor antagonists, 5-HT produces a monophasic cholinergically-mediated contraction, characterised by a pEC₅₀ of 9.2±0.06 (n=14). Increasing concentrations of SB-207266-A, (the HCl salt of SB207266) (10⁻¹⁰−10⁻⁸M, n=6) produced a parallel rightward shift of the 5-HT curve with no effect on the maximum response. The apparent pA₂ was 10.4±0.1, with a slope not significantly different from unity. At higher concentrations (3×10⁻⁸ M and above), the maximum response to 5-HT was reduced in a concentration-dependent manner. This effect of SB-207266-A was not due to a local anaesthetic action or to a direct antagonism at cholinergic receptors, since * DMPP-evoked contractions (a nicotinic receptor agonist which evokes acetylcholine release and hence a muscarinic receptor-mediated contraction) were unaffected even by a high concentration (10⁻⁵M) of the compound.

SB-207266-A was also tested against the contraction evoked by the 5-HT₄ receptor partial agonist BIMU 1. In these experiments, SB-207266-A reduced the maximum response to BIMU 1, without causing a prior right-ward shift in the concentration-response curve.

The apparent non-surmountable activity observed with SB-207266-A was not due to irreversible blockade of the 5-HT₄ receptor, since the antagonistic effects of SB-207266-A could be reversed upon washout. At the highest concentrations (which reduce maximum 5-HT-evoked contractions), responses to 5-HT recovered within 90 minutes. Such a profile is consistent with that of a reversible antagonist.

2) Piglet Atria

Compounds are tested in the piglet atria spontaneous beating screen (Naunyn-Schmiedeberg's Arch. Pharmacol 342, 619-622).

SB-207266-A (10⁻⁷M) shifted the curve to the right with an apparent reduction in the maximum response when compared to control curves with 5-HT alone. The estimated pK_{b} (−log₁₀ K_{b}) of SB-207266-A (SB 207266 as HCl salt) was 10.1 (n=2).

3) Rat Oesophagus

Rat oesophageal tunica muscularis mucosae is set up according to Baxter et. al. Naunyn-Schmiedeberg's Arch. Pharmacol., 343, 439-446 (1991). The inner smooth muscle tube of the muscularis mucosae is isolated and mounted for isometric tension recording in oxygenated (95% O₂/5% CO₂) Tyrodes solution at 37° C. All experiments are performed in pargyline pre-treated preparations (100 μM for 15 min followed by washout) and in the presence of cocaine (30 μM). Relaxant responses to 5-HT are obtained after pre-contracting the oesophagus tissue with carbachol (3 μM).

In the carbachol-contracted preparation, 5-HT produces concentration-dependent relaxations, with a pEC₅₀ of 8.1±0.03 (n=18). In contrast to the guinea-pig colon model, where the 5-HT₄ receptor is neuronally-located, the receptor here is located on the smooth muscle. In the rat oesophagus preparation, SB-207266-A concentration-dependently acted as a non-surmountable antagonist and reduced the maximum response evoked by 5-HT. Because SB-207266-A depressed the maximum response it was not possible to determine a reliable pA₂ estimate. However, the data obtained with the lowest effective concentration of SB-207266-A are consistent with a pA₂ of ≧10.0. In view of the high selectivity of SB-207266-A as a 5-HT₄ receptor antagonist (see previous guinea-pig isolated colon data and subsequent radioligand binding selectivity analysis), it is likely that the apparent non-surmountable antagonism is due to a slow dissociation of the compound from the receptor. This occurs because of the low 5-HT₄ receptor reserve in rat oesophagus and the high affinity of SB-207266-A at the 5-HT₄ receptor, relative to 5-HT itself.

4) Binding to Piglet Hippocampal 5-HT₄ Receptors

The affinity of SB-207266-A for piglet hippocampal 5-HT₄ receptors was determined from inhibition of binding of the ¹²⁵I-labelled 5-HT₄-antagonist SB-207710 [Brown A M, Young T J, Patch T L, Cheung C W, Kaumann A J, Gaster L M and King F D (1993), Br J Pharmacol; 110, 10P]. This radioligand has a high affinity for piglet hippocampal membranes (K_{D}=86±11 pM, Bₘₐₓ=16±3 fmol/mg protein (n=4)) while the pKᵢ's for SB-207710 are 6 or less at 5-HT_{1A}, 5-HT_{1C} and 5-HT₂ receptors. In addition, the 5-HT₃-selective ligand granisetron inhibits [¹²⁵I]-SB-207710 binding in hippocampus with a pKi of below 5, indicating negligible binding of the radioligand to 5-HT₃ receptors in this preparation. In this system, 5-HT binds to the 5-HT₄ receptor with a moderate affinity (pKi 6.6±0.1 (n=9)). SB-207266-A inhibited the binding of ¹²⁵I-labelled SB-207710 with a pKi value of 9.48±0.06 (n=3), a value slightly lower that the pA₂/pK_{B} estimates determined from antagonism of functional responses in other tissues.

5) Selectivity of SB-207266-A (SB 207266 as HCl Salt) in Vitro

SB-207266-A has been evaluated on a variety of non-5-HT₄ receptor binding assays. The results are shown in the Table below. Functional studies on the rat stomach fundus reveal an affinity for the 5-HT_{2B} receptor of 7.47. Clearly there are several orders of magnitude of selectivity for the 5-HT₄ receptor over the other receptors tested.

| Receptor Binding Studies | pK_{d} |
|---|---|
| 5-HT_{1A} | <5.00 |
| 5-HT_{1D} | <5.00 |
| 5-HT_{1E} | <5.00 |
| 5-HT_{2A} | 5.89 |
| 5-HT_{2C} | 5.57 |
| 5-HT₃ | 5.94 |
| Alpha₁ | <5.52 |
| D₂ | 5.63 |
| D₃ | 5.53 |
| GABA | >5.00 |
| BDZ | >5.00 |
| H₁ | 5.40 |
| Opiate kappa | (pKi) > 6 |
| Opiate mu | (pKi) > 6 |
| Opiate delta | (pKi) > 6 |

4) 5-HT-induced Motility in Dog Gastric Pouch

Compounds are tested for inhibition in the in vivo method described in “Stimulation of canine motility by BRL 24924, a new gastric prokinetic agent”, Bermudez et al, J. Gastrointestinal Motility, 1990, 2(4), 281-286.

Dogs with previously prepared Heidenhain gastric pouches are fasted overnight. For each dog, dose-ranging studies with 5-HT are also performed previously to ascertain the minimal intravenous (iv) dose which evokes a reproducible, cholinergically-mediated increase in tonic and phasic gastric contractility, usually 5 or 10 ug.kg⁻¹. For each experiment, 5-HT is administered iv at 30 min intervals. After two consistent responses, antagonists are injected iv or dosed po in a gelatine capsule 15 min before the third injection of 5-HT.

Both iv and po, SB-207266-A dose-dependently antagonised the contractile response to 5-HT [ID₅₀ 1.3 (Confidence Limits 0.1-14.0) ug.kg⁻¹ iv, 9.6 (CL 0.7-128) ug.kg⁻¹ po]. Furthermore there was no effect of SB-207266-A at any dose on basal motility. There was no consistent or significant effect with 5-HT₁, 5-HT₂ and 5-HT₃ receptor antagonists.

The duration of action of SB-207266-A was determined after iv dosing. At the lower doses of 1 and 3 ug.kg⁻¹ the effects were variable and apparently reversible, whilst at 10 and 100 ug.kg⁻¹, the antagonism lasted for more than the duration of the experiment (285 minutes).

5) Antagonism in Anaesthetised Piglets

In these experiments, antagonism is assessed against the 5-HT-evoked tachycardia, a response that is mediated by the 5-HT₄ receptors. All experiments were in 2-5 day old piglets in which the vagi were sectioned. SB-207266-A (SB 207266 as HCl salt) at doses of 0.1, 0.3 or 1.0 ugkg⁻¹ given intravenously antagonised the 5-HT-evoked tachycardia in a dose-dependent manner (n=2 each). At doses which substantially antagonise this 5-HT₄ receptor mediated effect of 5-HT (0.3, 1.0 ugkg⁻¹ i.v.), the recovery from antagonism was incomplete, over the duration of the experiment.

In Vivo Testing for Anxiolytic Activity

Social Interaction Test in Rats

Rats (male, Sprague Dawleys, Charles River, 250-300 g) are housed in groups of eight in a holding room for 5 days. They are then housed singly in a room adjacent to the experimental room for 4 days prior to the experimental day. On the experimental day rats are administered vehicle, test compound or a benzodiazepine anxiolytic, chlordiazepoxide, p.o. in pairs (n=8-16), at 15 minute intervals beginning at 10.00 a.m. 30 mins. later they are placed with a weight matched pair-mate (encountered for the first time) in the social interaction box in a separate room. The box is made of white perspex 54 cm×37 cm×26 cm with a transparent perspex front side and no lid. The floor is divided up into 24 squares and the box is brightly lit (115 lux). Active social interactive behaviours (grooming, sniffing, climbing over or under, following, biting, mounting and boxing) are scored blind for the next 15 min by remote video monitoring to give total interaction scores. The number of squares crossed by each rat is also scored and summed. After the end of each test the box is carefully wiped.

Significantly increased total interaction scores were observed 1 h after administration of SB-207266-A, (the HCl salt of SB 207266) (0.01, 1, 10 mg.kg⁻¹). The magnitude of this effect was somewhat smaller than that of the positive control chlordiazepoxide (CDP; 5 mg.kg⁻¹ po) but not significantly so. The effect of SB-207266-A was not accompanied by any alteration in locomotion during the test and hence is consistent with anxiolysis.

Tests Demonstrating that Prophylaxis/Treatment of Atrial Remodelling/Atrial Fibrillation Occurs via Antagonism of 5-HT₄ Receptors

If the skilled person wishes to demonstrate that the prophylaxes or treatments provided by some or all aspects of the invention occur via antagonism of 5-HT₄ receptors, one or both of the following tests (which are conventional and well known to the skilled person) can optionally be performed:
(1) Administer the 5-HT₄ antagonist to a mammal (e.g. dog) which naturally or artificially has no 5-HT₄ receptors expressed in its atrium (for example dogs naturally have no 5-HT₄ receptors expressed in their atrium). If atrial remodelling induced in the test animal is not at least partly reversed (and/or the AF incidence is not reduced or inhibited) by administration of the 5-HT₄ antagonist, then the prophylaxis or treatment of atrial remodelling (or AF) should occur via antagonism of 5-HT₄ receptors.
(2) Administer the 5-HT₄ antagonist using the the pig model shown in Examples 1 and/or 2, but in the presence of a sufficient amount of a 5-HT₄ receptor agonist such as cisapride. If the administered 5-HT₄ antagonist does not sucessfully reverse atrial remodelling at least partly e.g. by increasing AERP (and/or does not e.g. reduce AF incidence) then the prophylaxis or treatment of atrial remodelling (or AF) should occur via antagonism of 5-HT₄ receptors.

1-48. (canceled)

49. A method of treatment or prophylaxis of atrial arrhythmia in a mammal in need thereof, which comprises administering to said mammal an effective amount of N-[(1-ⁿbutyl-4-piperidinyl)methyl]-3,4-dihydro-2H-[1,3]oxazino[3,2-a]indole-10-carboxamide or a pharmaceutically acceptable salt thereof,
the method comprising administering the N-[(1-ⁿbutyl-4-piperidinyl)methyl]-3,4-dihydro-2H-[1,3]oxazino[3,2-a]indole-10-carboxamide or the salt thereof on the first day at a loading dose of about 1.2 to about 2.0 times the daily maintainance dose, and on subsequent days administering the N-[(1-ⁿbutyl-4-piperidinyl)methyl]-3,4-dihydro-2H-[1,3]oxazino[3,2-a]indole-10-carboxamide or the salt thereof at the daily maintainance dose.

50. A method as claimed in claim 49, wherein the loading dose is about 1.25 to about 2.0 times the daily maintainance dose.

51. A method as claimed in claim 49, wherein the loading dose is about 1.25 to about 1.75 times the daily maintainance dose.

52. A method as claimed in claim 49, wherein the loading dose is about 1.5 to about 2.0 times the daily maintainance dose.

53. A method as claimed in claim 49, wherein the loading dose is about 1.5 times the daily maintainance dose.

54. A method as claimed in claim 49, wherein the loading dose is about 2.0 times the daily maintainance dose.

55. A method as claimed in claim 49, wherein the daily maintenance dose comprises a daily oral or parenteral dosage regimen of about 0.2 mg to about 1.5 mg of the N-[(1-ⁿbutyl-4-piperidinyl)methyl]-3,4-dihydro-2H-[1,3]oxazino[3,2-a]indole-10-carboxamide or the pharmaceutically acceptable salt thereof per kg of total body weight (measured as the free base).

56. A method as claimed in claim 49, wherein the daily maintenance dose comprises a daily oral or parenteral dosage regimen of about 0.2 mg to 1.0 mg of the N-[(1-ⁿbutyl-4-piperidinyl)methyl]-3,4-dihydro-2H-[1,3]oxazino[3,2-a]indole-10-carboxamide or the pharmaceutically acceptable salt thereof per kg of total body weight (measured as the free base).

57. A method as claimed in claim 49, wherein the daily maintenance dose comprises a daily oral or parenteral dosage regimen of about 0.2 mg to about 0.5 mg of the N-[(1-ⁿbutyl-4-piperidinyl)methyl]-3,4-dihydro-2H-[1,3]oxazino[3,2-a]indole-10-carboxamide or the salt thereof per kg of total body weight (measured as the free base).

58. A method as claimed in claim 49, wherein the daily maintenance dose comprises a daily oral or parenteral dosage regimen of about 0.2 mg to 0.3 mg of the N-[(1-ⁿbutyl-4-piperidinyl)methyl]-3,4-dihydro-2H-[1,3]oxazino[3,2-a]indole-10-carboxamide or the salt thereof per kg of total body weight (measured as the free base).

59. A method as claimed in claim 49, wherein the daily maintenance dose comprises a daily oral or parenteral dosage regimen of about 0.5 mg to 1.0 mg of the N-[(1-ⁿbutyl-4-piperidinyl)methyl]-3,4-dihydro-2H-[1,3]oxazino[3,2-a]indole-10-carboxamide or the salt thereof per kg of total body weight (measured as the free base).

60. A method as claimed in claim 49, wherein the administration is to a human and wherein the daily maintenance dose comprises a daily oral or parenteral dosage of 20 mg of the N-[(1-ⁿbutyl-4-piperidinyl)methyl]-3,4-dihydro-2H-[1,3]oxazino[3,2-a]indole-10-carboxamide or the salt thereof (measured as the free base).

61. A method as claimed in claim 49, wherein the administration is to a human and wherein the daily maintenance dose comprises a daily oral or parenteral dosage of 50 mg of the N-[(1-ⁿbutyl-4-piperidinyl)methyl]-3,4-dihydro-2H-[1,3]oxazino[3,2-a]indole-10-carboxamide or the salt thereof (measured as the free base).

62. A method as claimed in claim 49, wherein the daily maintenance dose comprises a daily oral or parenteral dosage regimen of about 1.0 mg to about 1.5 mg of the N-[(1-ⁿbutyl-4-piperidinyl)methyl]-3,4-dihydro-2H-[1,3]oxazino[3,2-a]indole-10-carboxamide or the pharmaceutically acceptable salt thereof per kg of total body weight (measured as the free base).

63. A method as claimed in claim 49, wherein the daily maintenance dose comprises a daily oral or parenteral dosage regimen of 1.0 mg to 1.3 mg of the N-[(1-ⁿbutyl-4-piperidinyl)methyl]-3,4-dihydro-2H-[1,3]oxazino[3,2-a]indole-10-carboxamide or the pharmaceutically acceptable salt thereof per kg of total body weight (measured as the free base).

64. A method as claimed in claim 49, wherein the administration is to a human and wherein the daily maintenance dose comprises a daily oral or parenteral dosage of 80 mg of the N-[(1-ⁿbutyl-4-piperidinyl)methyl]-3,4-dihydro-2H-[1,3]oxazino[3,2-a]indole-10-carboxamide or the salt thereof (measured as the free base).

65. A method as claimed in claim 52, wherein the administration is to a human and wherein the daily maintenance dose comprises a daily oral or parenteral dosage of 20 mg of the N-[(1-ⁿbutyl-4-piperidinyl)methyl]-3,4-dihydro-2H-[1,3]oxazino[3,2-a]indole-10-carboxamide or the salt thereof (measured as the free base).

66. A method as claimed in claim 52, wherein the administration is to a human and wherein the daily maintenance dose comprises a daily oral or parenteral dosage of 50 mg of the N-[(1-ⁿbutyl-4-piperidinyl)methyl]-3,4-dihydro-2H-[1,3]oxazino[3,2-a]indole-10-carboxamide or the salt thereof (measured as the free base).

67. A method as claimed in claim 52, wherein the administration is to a human and wherein the daily maintenance dose comprises a daily oral or parenteral dosage of 80 mg of the N-[(1-ⁿbutyl-4-piperidinyl)methyl]-3,4-dihydro-2H-[1,3]oxazino[3,2-a]indole-10-carboxamide or the salt thereof (measured as the free base).

68. A method as claimed in claim 53, wherein: the mammal is a human; the loading dose is 30 mg, 75 mg or 120 mg; and the daily maintainance dose is 20 mg, 50 mg or 80 mg respectively.

69. A method as claimed in claim 49, wherein the administration, dosage and/or dosage regimen of the N-[(1-ⁿbutyl-4-piperidinyl)methyl]-3,4-dihydro-2H-[1,3]oxazino[3,2-a]indole-10-carboxamide or the salt thereof is oral.

70. A method as claimed in claim 52, wherein the administration, dosage and/or dosage regimen of the N-[(1-ⁿbutyl-4-piperidinyl)methyl]-3,4-dihydro-2H-[1,3]oxazino[3,2-a]indole-10-carboxamide or the salt thereof is oral.

71. A method as claimed in claim 65, wherein the administration, dosage and/or dosage regimen of the N-[(1-ⁿbutyl-4-piperidinyl)methyl]-3,4-dihydro-2H-[1,3]oxazino[3,2-a]indole-10-carboxamide or the salt thereof is oral.

72. A method as claimed in claim 66, wherein the administration, dosage and/or dosage regimen of the N-[(1-ⁿbutyl-4-piperidinyl)methyl]-3,4-dihydro-2H-[1,3]oxazino[3,2-a]indole-10-carboxamide or the salt thereof is oral.

73. A method as claimed in claim 67, wherein the administration, dosage and/or dosage regimen of the N-[(1-ⁿbutyl-4-piperidinyl)methyl]-3,4-dihydro-2H-[1,3]oxazino[3,2-a]indole-10-carboxamide or the salt thereof is oral.

74. A method as claimed in claim 49, wherein the medicament, method, or compound is for or employs administration of the daily maintainance dose for from about 1 week up to the mammal's entire remaining life.

75. A method as claimed in claim 49, wherein the atrial arrhythmia comprises atrial fibrillation.

76. A method as claimed in claim 49, wherein the mammal is a sufferer of or susceptible to persistent atrial fibrillation.

77. A method as claimed in claim 49, wherein the mammal is a sufferer of or susceptible to paroxysmal atrial fibrillation.

78. A method as claimed in claim 49, wherein the method is for, of, or for use in the inhibition of symptomatic recurrences of atrial fibrillation in a mammal with paroxysmal or persistent atrial fibrillation.

79. A method as claimed in claim 49,
wherein the method is for or employs administration of the loading dose during an arrhythmic episode in the mammal, and
wherein the method is for or employs administration of the maintenance dose after cardioversion of the mammal back to normal sinus rhythm, the cardioversion being done in the event that the mammal is not in normal sinus rhythm after a period sufficient for the loading dose to take effect.

80. A method as claimed in claim 79, wherein the period sufficient for the loading dose to take effect is about 1 to about 8 hours.

81. A method as claimed in claim 79, wherein the arrhythmic episode is an atrial fibrillatory episode.

82. A method as claimed in claim 49, wherein the mammal is a human.

83. A method as claimed in claim 70, wherein the mammal is a human.

84. A method as claimed in claim 71, wherein the mammal is a human.

85. A method as claimed in claim 72, wherein the mammal is a human.

86. A method as claimed in claim 73, wherein the mammal is a human.

87. A method of treating a mammal who is experiencing an arrhythmic episode, comprising:
(a) administering N-[(1-ⁿbutyl-4-piperidinyl)methyl]-3,4-dihydro-2H-[1,3]oxazino[3,2-a]indole-10-carboxamide or a pharmaceutically acceptable salt thereof at a loading dose as defined in claim 49,
(b) waiting for a period sufficient for the dose in step (a) to take effect at least partially,
(c) optionally measuring whether the mammal has reverted to normal sinus rhythm,
(d) cardioverting the mammal back to normal sinus rhythm in the event that the mammal is not in normal sinus rhythm after the period in step (b), and then
(e) administering as necessary the N-[(1-ⁿbutyl-4-piperidinyl)methyl]-3,4-dihydro-2H-[1,3]oxazino[3,2-a]indole-10-carboxamide or the salt thereof at the daily maintenance dose on subsequent days as defined in claim 55.

88. A method as claimed in claim 87, wherein the period in step (b) is about 1 to about 8 hours.

89. A method as claimed in claim 87, wherein the period in step (b) is about 1 to about 4 hours.

90. A method as claimed in claim 87, wherein in step (d) direct current (DC) cardioversion is used.
